(19) United States
(12) Patent Application Publication
(10) Pub. No.: US 2025/0366750 A1
(43) Pub. Date: Dec. 4, 2025

(54) Determination of physiological state based on analysis of metrics derived from patient heart and brain waveforms

(71) Applicant: Medibio Limited, Savage, MN (US)

(72) Inventors: Archie Defillo, Savage, MN (US); Massimiliano Grassi, Milan (IT)

(21) Appl. No.: 18/874,300

(22) PCT Filed: Jun. 14, 2023
(86) PCT No.: PCT/US2023/025291

§ 371 Date: Dec. 12, 2024

Related U.S. Application Data
(60) Provisional application No. 63/352,031, filed on Jun. 14, 2022.

(51) Int. Cl. A61B 5/16; A61B 5/00; A61B 5/308; A61B 5/31; A61B 5/347; A61B 5/349; A61B 5/374; G16H 20/70; G16H 50/20; G16H 50/30

(52) U.S. Cl. CPC A61B 5/165; A61B 5/308; A61B 5/31; A61B 5/347; A61B 5/349; A61B 5/374; A61B 5/4812; A61B 5/7267; G16H 20/70; G16H 50/20; G16H 50/30

(57) ABSTRACT

In general, the subject matter described in this disclosure can be embodied in methods, systems, and program products for identifying a value of a heart rate variability metric that indicates a variation in a heart waveform of a patient; identifying a value of a brain activity metric that indicates a type of electrical activity represented by a brain waveform of the patient; providing values for a collection of metrics to a computational model, the values for the collection of metrics including the value for the heart rate variability metric and the value for the brain activity metric; and receiving, from the computational model as a result of having provided the values for the collection of metrics to the computational model, an indication of mental state of the patient.

### TECHNICAL FIELD

This document generally relates to biometric measurement and analysis.

### BACKGROUND

Various types of mental states of patients are diagnosed by clinical assessment and opinion, based on interviews with patients and completion of questionnaires by patients. Many aspects of diagnostic processes are subjective, and assessment can vary between clinicians.

### SUMMARY

This document describes techniques, methods, systems, and other mechanisms for determining a behavioral health state of a patient based on analysis of heart and brain waveforms of the patient.

In general, a computing system can receive data that represents, for each of multiple patients: (1) a heart waveform of the respective patient, (2) a brain waveform of the respective patient, and (3) an indication of mental state of the respective patient; derive biometric parameters based on the waveforms; and use the biometric parameters to generate a computational model that represents relationships between a mental state and the biometric parameters.

The same biometric parameters can be derived for a patient for which their mental state is unknown, and the same biometric parameters can be provided to the computational model. The computational model can use the biometric parameters to determine a likely mental state of the patient, based on the relationships represented by the computational model.

An example biometric parameter includes variability in heart rate of a patient. The variability in heart rate can be a variation in lengths of heart beats in a heart waveform (e.g., variation among a set of consecutive heart beats, with the length of each heart beat measured from a peak of one “R” wave to a peak of a next “R” wave). The variability in heart rate of the patient can also or additionally represent a variability in frequencies present in the heart waveform during a period of time (e.g., based on a Fast Fourier Transform of a portion of the heart waveform, or a Fast Fourier Transform of a series of lengths of heart beats represented by the portion of the heart waveform).

Another example biometric parameter can represent variability in brain states of a patient. The variability in brain states can be a variability in frequencies exhibited by a brain waveform of the patient.

These biometric parameters, and others, are described in additional detail throughout this application. The actual combination of biometric parameters on which a particular computational model is configured can vary, and include one or more such parameters, and/or parameters derived therefrom. An example derived parameter is a difference between a same parameter over two different sleep stages (e.g., a level of time-domain heart rate variability during N3 sleep compared to a level of time-domain heart rate variability during REM sleep). Another example, derived parameter is a difference between different parameters during the same sleep stage (e.g., a level of Alpha brain waves during N2 sleep compared to a level of Beta brain waves during N2 sleep). Another example derived parameters is a coupling of two parameters during different sleep stages (e.g., how an Alpha-to-Beta ratio of brain waves during N3 sleep compares to the Alpha-to-Beta ratio of brain waves during REM sleep.

Particular implementations can, in certain instances, realize one or more of the following advantages. A computational model that is able to classify likely mental states of patients based on biometrics that are derived from physical characteristics of the patients can provide for objective characterization of patient mental states. Such technology can be less expensive than individual-by-individual assessment by clinicians, and can therefore provide for more widespread testing of potential mental states, to support clinician referral and earlier diagnosis. This technology can therefore assist clinicians by enabling them to spend less time on testing and more time on therapy.

Technology that can be used to determine mental states of patients can provide for objective comparison of therapeutic effectiveness over time, which can enable clinicians to vary and optimize treatments based on measured responses to therapies. Such technology not only can improve patient treatment and outcomes, but can provide savings to health systems and patients.

The details of one or more implementations are set forth in the accompanying drawings and the description below. Other features, objects, and advantages will be apparent from the description and drawings, and from the claims.

### DESCRIPTION OF DRAWINGS

FIGS. 1A-B show a diagram of a system for determining a physiological state of a patient.

FIGS. 2A-B illustrate details of a sleep stage classification process performed by the sleep stage determiner.

FIG. 3 illustrates a classification of a sleep episode into sleep stages.

FIG. 4 shows a time-domain heart rate variability metric determiner.

FIG. 5 shows a frequency-domain heart rate variability metric determiner.

FIG. 6 shows a brain state metric determiner.

FIGS. 7A-B show other metric determiners.

FIGS. 8A-B multiple example collections of metrics.

FIG. 9 is a conceptual diagram of a system that may be used to implement the systems and methods described in this document.

FIG. 10 is a block diagram of computing devices that may be used to implement the systems and methods described in this document, as either a client or as a server or plurality of servers.

Like reference symbols in the various drawings indicate like elements.

### DETAILED DESCRIPTION

This document describes techniques, methods, systems, and other technologies for determining a behavioral health state of a patient based on analysis of heart and brain waveforms of the patient. The determination of the behavioral health state of the patient can be a determination of a mental state of the patient based on physiological manifestation of that mental state over a period of time (e.g., a sleep session), as determined based on analysis of heart and brain waveforms of the patient.

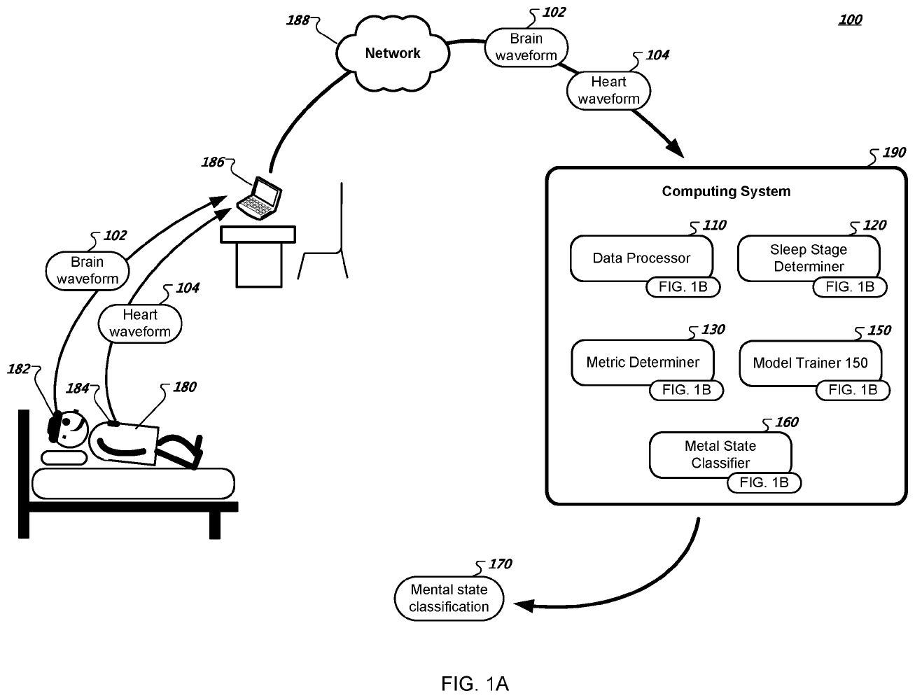

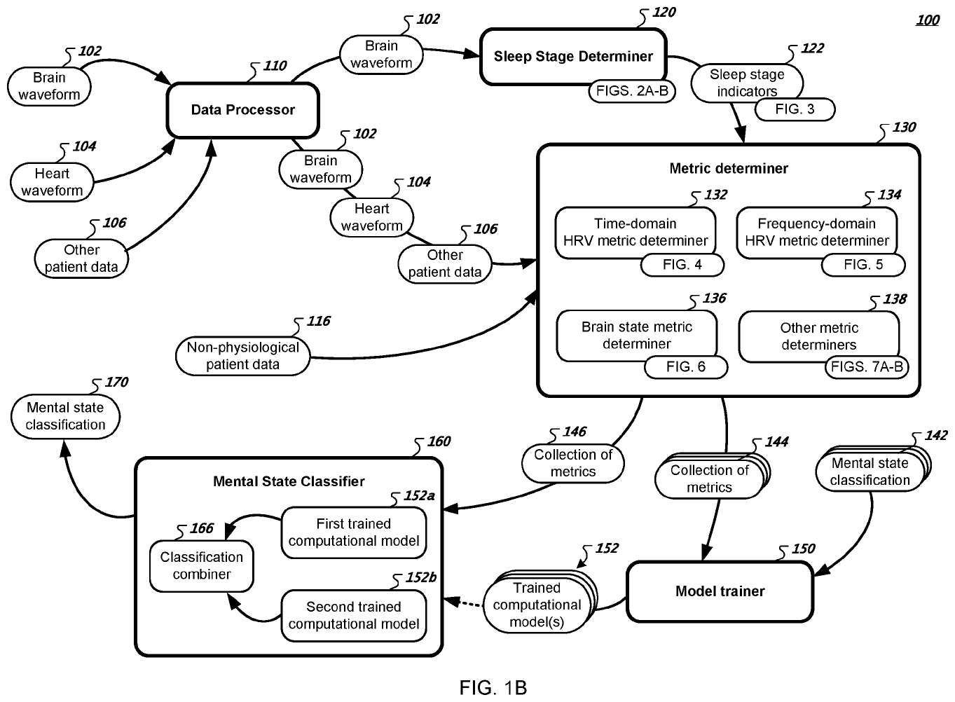

FIG. 1A shows a high-level overview of a system 100 that determines a behavioral health state of a patient. A patient 180 participating in a sleep study may wear: (1) a cap 182 that includes electrodes for recording brain waveform data 102 (e.g., an electroencephalogram (EEG)); and (2) a chest-worn device 182 that is able to record a heart waveform 184 (e.g., an electrocardiogram (ECG)). These devices may transmit the physiological data to a user device 186, which may transmit the physiological data over a network 188 to a server system 190. The server system 190 includes various components 110, 120, 130, 150, and 160 that processes the data to generate a mental state classification 170, which can classify brain and heart activity of a patient as exhibiting features present in patients previously diagnosed as exhibiting a particular mental state (e.g., depression).

FIG. 1B shows additional detail regarding components 110, 120, 130, 150, and 160 of system 100. The system 100 includes a data processor 110 that processes the brain waveform data 102 and the heart waveform. For example, the data processer 110 may receive the EEG and the ECG signals and remove noise from the data signals.

A sleep stage determiner 120 analyzes the brain waveform 102 to classify a patient sleep session into different stages of sleep (e.g., awake, N1 sleep, N2 sleep, N3 sleep, and REM sleep). The system 100 may designate various portions of the brain waveform 102 and the heart waveform 104 as having occurred during the various stages of sleep (e.g., designate a thirteen-minute portion of the brain waveform 102, or data determined therefrom, as having been recorded during REM sleep).

A metric determiner 130 determines various different types of metrics specific to a particular patient. The metrics may be generated from brain waveform 102, the heart waveform 104, and other data to form a collection of metrics 144 that represent characteristics of a respective patient. The system 100 may perform such operations on each of multiple patients, generating values for a collection of metrics 144 for each of multiple patients from whom data is used to train a computational model. The system 100 may also access a mental state classification 142 for each of the multiple patients from whom data is used to train the computational model.

A model trainer 160 receives: (1) the collection of metrics 144 for each of multiple patients; and (2) the mental state classification 142 for each of the multiple patients; and generates therewith one or more trained computational models 152.

A mental state classifier 160 may use the one or more trained computational models 152 to classify a mental state 170 of a patient for whom mental state is unknown. For example, system 100 can: (1) receive and EEG and ECG from a patient whose mental state is unknown, (2) process that data using the sleep stage determiner 120 and the metric determiner 130 to determine values for a collection of metrics 146 specific to the patient, and (3) provide the values for the collection of metrics 146 to the mental state classifier 160, which applies the collection of metrics to the one or more trained models 152a-b to generate a mental state classification 170 for the patient.

System 100 can generate a computational model that is able to classify probable mental states of patients based on biometrics. This technology provides for objective characterization of patient mental states, enabling clinicians to focus more on therapy and providing clinicians with an additional tool to diagnose patient mental states.

Now describing system 100 in additional detail, the data processor 110 receives various types of patient data and processes that data. Two example types of patient data include brain waveform data 102 and heart waveform data 104. The brain waveform data 102 and the heart waveform data 104 may have been recorded from a single patient during a single sleep session (e.g., a single night) or over multiple sleep sessions (e.g., multiple nights).

In some examples, the brain waveform data 102 is an electroencephalogram (EEG) acquired using one or more electrodes attached to the patient during the sleep study (e.g., using six EEG montages: C4A1, F4A1, 02A1, F3A2, C3A2, and O1A2). The EEG may be obtained by individual electrodes attached to a scalp of the patient or a headset that incorporates such electrodes. In some examples, the brain waveform data 102 is acquired by a sensor contained in a consumer device headset worn by the patient during sleep at home.

In some examples, the heart waveform data 104 is an electrocardiogram (ECG) acquired using one or more electrodes attached to the patient during the same sleep study (e.g., using a Lead Il electrode arrangement). In some examples, heart waveform data 104 is obtained alternatively or additionally through the use of a wrist-based sensor configured to detect wrist pulse (e.g., an optical-based system embedded in a watch-like device that attaches at a patient's wrist). In such examples, the heart waveform data 104 may represent blood flow waveform data and may not directly represent heart electrical activity. In some examples, heart waveform data 104 is obtained alternatively or additionally through a movement or electrical-activity sensor located at a patient's chest, to record heart movement or electrical activity at the patient's chest.

In some examples, other patient data 106 is acquired by additional or alternative sensors worn by the patient. For example, the other patient data 106 may include any combination of: a chin electromyogram (EMG), a leg EMG electrode recording, an electrooculography reading, weight data obtained from a weight scale, respiratory data obtained by a respiratory sensor that analyzes a patient's respiration, and/or physical activity data obtained by a physical activity sensor that analyzes levels of patient activity over time. The physical activity sensor can include one or more accelerometers and/or gyroscopes incorporated into a wearable device, such as a wrist-mounted watch that may additionally include sensors to record the heart waveform 104.

The brain waveform data 102, the heart waveform data 104, and other data 106 may be recorded by a single device or different devices, for example, during a single sleep session.

The data processor 110 receives one or more of the above described signals (e.g., one or more of a brain waveform 102, a heart waveform 104, weight data, respiratory data, and physical activity data) and processes the data, for example, by digitizing and/or filtering such signals. For example, the data processor 110 may filter a recording of an EEG brain waveform to remove noise from the EEG brain waveform. Similarly the data processor 110 may filter an ECG heart waveform.

The data processor 110 (and each of the other components 120, 130, 150, and 160 illustrated in FIG. 1) represents operations of one or more algorithms encoded by computer-readable media and executable by one or more processors, and can be located at a patient-interfacing device, a device remote from the patient (e.g., at a cloud computing system), a computing system hosted by a clinician, or distributed among a combination of multiple such systems. For example, a patient that is undergoing a sleep study may have the brain waveform data 102 and the heart waveform data 104 collected by one or more computerized devices present at a location of the sleep study. and the one or more computerized devices may perform the operations of the data processor 110.

As another example, the brain waveform data 102 and the heart waveform data 104 may be sent to a cloud computing system that performs the operations of the data processor 110. In examples in which patient biometric data is collected by a patient-worn device (e.g., a wrist-worn device), the data processing may be performed by the patient-worn device, by a patient device in communication with the patient-worn device (e.g., a connected smartphone), or by a device remote from the patient (e.g., a could computing system).

Patient physiological data, before and/or after data processing, may be accompanied by identifying metadata, such as a patient identifier (e.g., a numerical code that represents the patient) and/or a device identifier (e.g., an identifier of a monitoring device that includes the corresponding sensor(s), or a linked device in communication therewith, such as a smartphone).

System 100 may also process and analyze non-physiological patient data 116, such as user-entered data that specifies characteristics regarding a patient. For example, before or after a patient participates in a sleep study, the patient may answer questions regarding information that describes characteristics of the patient. Example types of information that is answered by the patient or that addresses a state of the patient include: (1) patient characteristics (e.g., age, height, weight, sex, ethnicity, body mass index); (2) medical symptoms experienced by the patient at a time that biometric data is gathered (e.g., body temperature, coughing, sneezing, bloating, nausea); (3) dietary information (e.g., food or drink consumed, alcohol use); (4) medicines taken by the patient; (5) possible mental states experienced by the patient (e.g., depression, anxiety, schizophrenia); (6) perceived emotional states experienced by the patient (e.g., happy, sad, anxious, tired); and (7) a level of physical activity of the patient (e.g., an amount of exercise a week).

In some examples, the system 100 can generate non-physiological patient data 116 from clinical documents. For example, a sleep clinic may generate documents that indicate results of a polysomnography sleep study. The clinical documents may be received by system 100 in PDF or image format, and system 1000 may process the documents to generate metrics from the clinical documents. Example metrics include any combination of one or more of: (1) an Apnea-Hypopnea Index (from a whole sleep session, a particular sleep stage, or type of sleep stage (e.g., REM sleep or non-REM sleep)); (2) a number of various types of arousals (from a whole sleep session, a particular sleep stage, or type of sleep stage (e.g., REM sleep or non-REM sleep)); (3) SpO2 (e.g., average or minimal); (4) a number of snoring episodes; (5) time in any one or more sleep stages, based on technician sleep staging; (6) demographic information (e.g., age, sex); and (7) clinical information (e.g., medications, comorbidities, body mass index (BMI)). As such, some such metrics relate to physiological parameters, but they are derived by system 100 from clinical documents rather than sensor measurements. Some non-physiological patient data 116 is not derived by any system from sensor measurements (e.g., age, sex, medications, comorbidities, BMI).

In some examples, system 100 provides the documents to a large language model to extract the required information and store that information in a object format recognized by system 100 (e.g., a Python dictionary or vector). Using information from such documents enables system 100 to generate metrics from not only the results of the polysomnography, but also any descriptors placed into the report by a clinician that performed, supervised, or analyzed the polysomnography. (e.g., pdf or image formats)

The patient may enter such answer to such questions into a computing device (e.g., a handheld tablet computing device), or the patient may provide the answers to another person (e.g., a sleep center employee) that may him or herself enter the answers into a computer. In either event, such non-physiological patient data 116 is provided to and received by system 100, for analysis.

The sleep stage determiner 120 determines a state of sleep of a patient at different times during a sleep session. For example, the sleep stage determiner may analyze the brain waveform 102 every thirty seconds and use a trained machine learning pipeline to analyze and classify the thirty-second portion of the brain waveform 102 as failing into one of multiple stages of sleep, such as Awake (not asleep), N1 (light transitional sleep), N2 (more stable sleep), N3 (deep sleep), and REM (revitalizer memory sleep).

The determination of a sleep stage for a given period of time may be based on a brain state of the patient, as exhibited by the brain waveform 102. For example, a high level or proportion of Beta band activity can indicate that the patient is awake, a high level or proportion of Alpha band activity can indicate that the patient is in N1 sleep, a high level or proportion of Delta band activity can indicate that the patient is in N3 sleep, and a high level or proportion of saw tooth activity can indicate that the patient is in REM sleep.

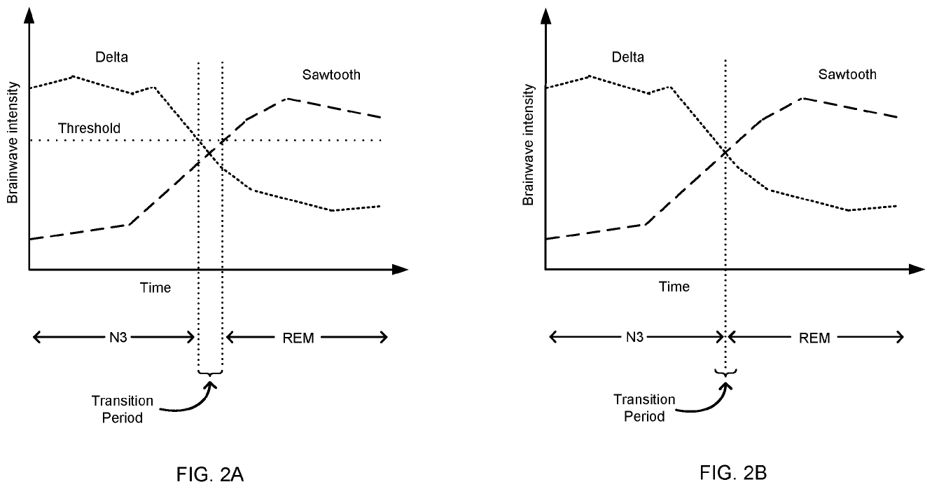

FIGS. 2A-B illustrate examples of how the sleep stage determiner 120 determines a sleep stage of a patient. In some examples, the sleep stage determiner 120 classifies a patient as being in a particular sleep stage as a result of a particular band of brain wave frequencies that is correlated with the particular sleep stage satisfying certain criteria.

In some examples, the criteria is the particular band of brain wave frequencies exceeding a threshold level (e.g., 20% or more of brain wave activity is of the particular band). FIG. 2A illustrates that the sleep stage determiner 120 has identified intensities of various brainwave frequency bands at different moments in time (with FIG. 2A showing only Delta and Sawtooth frequency bands, for ease of illustration). In the FIG. 2A example, the sleep stage determiner 120 has classified a first portion of a sleep session as N3 sleep based on a level of Delta frequency band activity exceeding a threshold, and has classified a second portion of the sleep session as REM sleep based on a level of Sawtooth frequency band activity exceeding the threshold.

In the FIG. 2A example, the sleep stage determiner 120 classified a portion of the sleep session between the N3 and REM sleep stages as a transition period, as a result of the Delta and Sawtooth frequency band activity both not exceeding the threshold for the corresponding amount of time. The system 100 may analyze biometrics recorded by system 100 during the transition period separate from biometrics recorded during the sleep periods, as described in additional detail later.

In some examples, the threshold level is different for each frequency band. For example, Delta band activity may need to exceed 20% for the sleep stage determiner 120 to classify sleep as N2 sleep, while Sawtooth band activity may need to exceed 30% for the sleep stage determiner 120 to classify sleep as REM sleep. In some examples, when multiple brainwave bands exceed their respective thresholds, the sleep stage determiner 120 may classify sleep as being in the stage associated with a greatest level of brainwave activity.

In some examples, the criteria to classify a patient as being in a particular sleep stage includes a particular frequency band of brain waves correlated with the particular sleep stage providing a highest level of activity among various brainwave bands. For example, FIG. 2B illustrates how the sleep stage determiner 120 classified (1) a first portion of sleep as N3 sleep based on a level of Delta frequency band activity exceeding a level of Sawtooth frequency band activity (and all other bands); and (2) a second portion of the sleep as REM sleep based on a level of Sawtooth frequency band activity exceeding the level of Delta frequency band activity (and all other bands).

In some examples, the sleep stage determiner 120 classifies a portion of patient sleep around a change from one sleep stage to another as as a transition period. For example, the sleep stage determiner 120 may classify a portion of sleep preceding, following, or straddling the identified moment of change from N3 to REM sleep as a transition period. In some examples, the transition period is distinct from the adjacent sleep stages (e.g., such that the N3 and REM sleep stages do not run concurrent with the transition period, as illustrated in FIG. 2A). In some examples, the transition period overlaps with the adjacent sleep stages (e.g., such that an end of the N3 sleep stage overlaps with a first portion of the transition period, and a beginning of the REM sleep stage overlaps with a second portion of the transition period, as illustrated in FIG. 2B).

As described above, the sleep stage determiner 120 may classify each thirty-second period of patient sleep into a sleep stage, with this thirty-second period being referred to as an “epoch” of time. Still, the epoch may be lengths of time other than 30 seconds, such as 10 seconds, 1 minute, or 5 minutes. The length of epochs during which a particular type of sensor data is analyzed may remain the same over a sleep session. For example, an entire sleep session of brain waveform data 102 may be broken up into 30 second epochs that are each classified as exhibiting a single type of sleep stage.

Each sleep stage determination may be stored by the system 100 with an accompanying absolute or relative timestamp, to enable system 100 to correlate sleep stages to corresponding portions of sensor data collected over a sleep session. As such, the system 100 is able to correlate portions of the brain waveform 102, the heart waveform 104, and the other patient data 106 with each other and with determined sleep stages.

The sleep stage determiner 120 may operate and perform such determinations at a patient device (e.g., a smart watch), a clinician device (e.g., a computer at a sleep center), and/or at a remote computing system (e.g., a cloud computing system).

In some implementations, the sleep stage determiner 120 may alternatively or additionally make its sleep stage determinations based on one or more of heart waveform data 104 or a metric derived therefrom (e.g., heart beats per minute), manual notifications (e.g., by a clinician at a sleep center), and activity data (e.g., movement measured by a wrist-worn device).

In some examples, the sleep stage determiner 120 may include a computational model that has been trained to classify sleep into stages based the intensity of various EEG bands. The computational model may have been trained based on multiple sets of patient EEG data classified into various sleep stages.

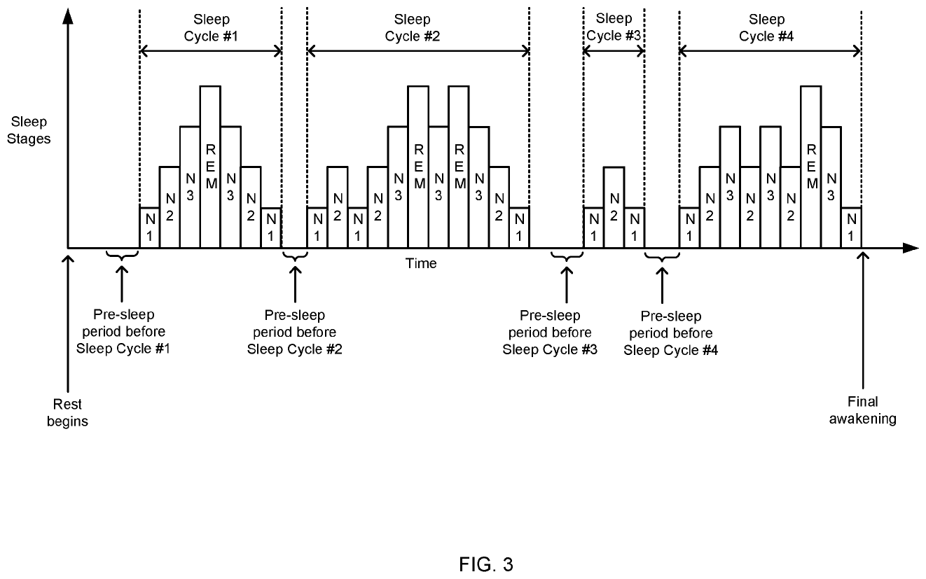

FIG. 3 shows a diagram that illustrates how the sleep stage determiner 120 categorizes an example sleep session into various sleep stages. The diagram of FIG. 3 graphically represents the sleep stage indicators data 122, illustrated in FIG. 1 as generated by the sleep stage determiner 120 and provided to the metric determiner 130. The FIG. 3 diagram illustrates a single sleep session, from a moment that a patient laid down to sleep (at left) to a moment that a patient awoke from sleep a final time (at right). Each vertical bar represents a classified sleep stage for a particular portion of sleep, with no bar indicating that the patient was classified as having been awake at the moment.

FIG. 3 illustrates four distinct sleep cycles during the sleep session, separated by periods in which the patient was momentarily awake. Sleep Cycle #1 illustrates a sleep cycle in which the patient progressed in a sequential manner from N1 sleep to REM sleep and back to N1 sleep, before awakening. Sleep Cycle #2 illustrates a more-complex sleep cycle, in which the patient alternated between N1 and N2 sleep stages before proceeding to REM sleep, and alternated between N3 and REM sleep before proceeding back to N1 sleep and then awakening. Sleep Cycle #3 illustrates a shorter sleep cycle, in which the patient only reached N2 sleep before falling back to N1 sleep and awakening. Sleep Cycle #4 illustrates a sleep cycle in which the patient entered REM sleep twice, skipping the N3 sleep stage transition when transitioning to the REM sleep stage and skipping the N2 sleep stage transition when transitioning to the final N1 sleep stage.

FIG. 3 illustrates how the sleep stage determiner 120 may classify a portion of the sleep session before the patient entered each sleep cycle as a pre-sleep period. The system 100 can analyze patient biometrics during this pre-sleep period separately from the sleep stages, because patients with mental disorders can exhibit unique biometric characteristics during the pre-sleep period. In some examples, the sleep stage determiner 120 classifies the pre-sleep period as a duration of fixed period of time before a sleep cycle (e.g., a two-minute period before falling asleep, as illustrated by Sleep Cycle #1). In some examples, the sleep stage determiner 120 classifies an entire awake period before a sleep cycle as a pre-sleep period, as illustrated by Sleep Cycle #4.

FIG. 3 represents a simplified representation of a sleep session, for illustration purposes, and an actual representation of a sleep session is likely to differ. For example, FIG. 3 illustrates each sleep stage as a same length, but lengths of sleep stages may differ during a night. FIG. 3 also does not illustrate transition periods between the various sleep stages, but the sleep stage determiner 120 may have classified portions of the sleep session as transition periods.

The metric determiner 130 receives various types of data and generates values for a collection of metrics therefrom (e.g., with the values being for the collection of metrics 144 during training operations, and being for the collection of metrics 146 during runtime operations). The collection of metrics may be a collection of numbers that each represent a biometric statistic that indicates an amount of a physiological characteristic of the patient. The data received by the metric determiner and from which the values for the metrics are determined can include at least: (1) the sleep stage indicators 122, and (2) one or more of the brain waveform 102, the heart waveform 104, the other patient physiological data 106 (e.g., respiratory data), and the patient non-physiological data 116 (e.g., the above-discussed patient answers to one or more questionnaires and/or the patient data from clinical documents).

As with the sleep stage determiner 120, the metric determiner 130 and its components may operate and perform their operations at a patient device (e.g., a smart watch), a clinician device (e.g., a computer at a sleep center), and/or at a remote computing system (e.g., a cloud computing system). The metric determiner 130 receives data from the data processor 110 and the sleep stage determiner 120 (communicating from one computing device to another as needed to function across multiple devices, should the components be implemented by different computing devices).

As discussed throughout this disclosure, during runtime operation the metric determiner 130 generates values for the collection of metrics 146 from data of a particular patient, and those values are provided to the mental state classifier 160 to classify a mental state of the particular patient. Values for the same collection of metrics are generated for multiple (e.g. thousands) of patients during operations that train the computational model to be used during runtime operation. The collection of metrics used during training and runtime operation may include multiple different metrics, at least some of which are generated based on the brain waveform data 102 and/or the heart waveform data 104.

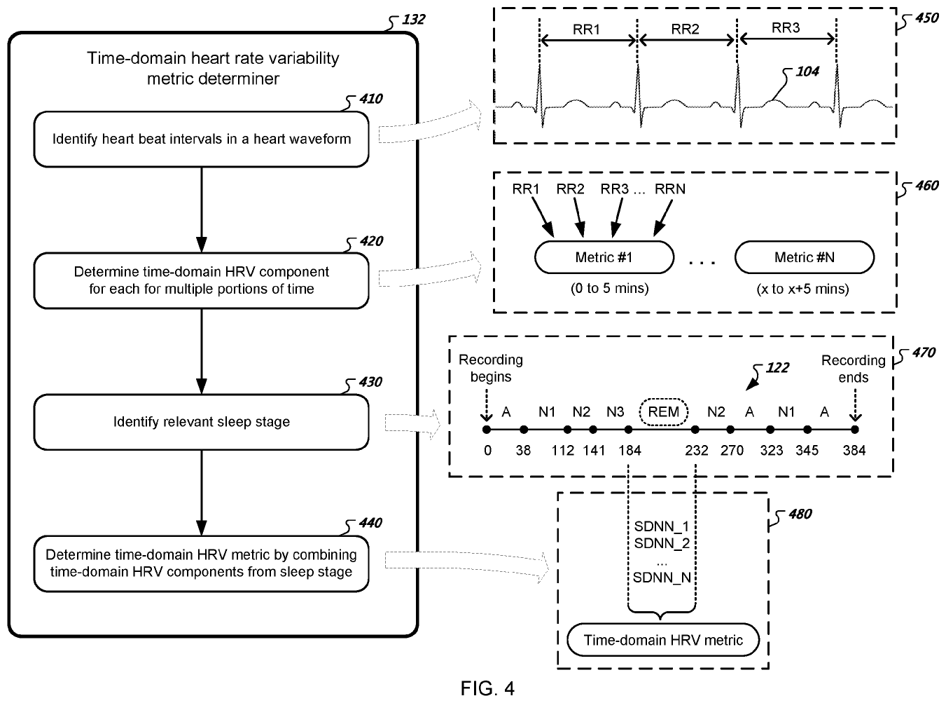

The metric determiner 130 of FIG. 1 includes four sub-components 132, 134, 136, and 138 that generate example metrics, some of which may be used in or form the basis for other metrics included in the collection of metrics 146. These example metrics are discussed in additional detail below, with reference to FIGS. 4-7.

FIG. 4 shows the time-domain heart-rate variability (HRV) metric determiner 132 of FIG. 1. with additional detail and illustration. The time-domain HRV metric determiner 132 can perform the operations of boxes 410-440 shown in FIG. 4 to generate a time-domain HRV metric that may be, or be used in the formation of, a metric included in the collection of metrics 144, 146.

At box 410, the computing system on which the time-domain HRV metric determiner 132 is operating can receive heart waveform data 104 and identify heart beat intervals therefrom. For example, the computing system can process the heart waveform 104 and determine a starting location of each heart beat or a length of each heart beat, from one instance of a heart beat feature to another instance of the heart beat feature (e.g., from one instance of an R wave peak to another instance of the R wave peak, even though an R wave is a portion of a heart beat rather than a dividing feature between successive heart beats). Item 450 illustrates an identification of lengths of successive RR intervals (e.g., RR1, RR2, RR3) in the heart waveform 104. Artifacts in an ECG signal can lead to an incorrect RR interval determination. As a result, the system may apply artifact detection algorithms to exclude potentially incorrect RR intervals from the data. Illegitimate RR intervals that are generated by a premature ventricular contractions or other type of arrhythmia may be identified and excluded from the data.

At box 420, the computing system determines the time-domain HRV component for each of multiple portions of time. For example, the computing system may calculate a metric that represents variation among heart beat intervals over a given period of time. For example, the computing system may determine the standard deviation of lengths of RR intervals (SDNN) for heart beat intervals occurring within a five minute epoch. This computing system may calculate this SDNN metric every five minute epoch, repeatedly over an entire sleep session or a designated portion of a sleep session (e.g., one or more designated sleep stages).

Item 460 illustrates how the system combines heart rate intervals over a given time period (e.g., RR1-RRN over a 5-minute interval) into a single metric that represents time-domain variation of heart rate intervals over the given time period. This process can repeat for each such time period (e.g., every 5-minute interval). The system 100 can generate the metric in real time during a sleep session as the heart waveform is sensed and processed, or after the sleep session during a post processing of sleep data.

At box 430, the computing system identifies the relevant sleep stage for which a metric is to be generated and selects data for the relevant sleep stage. For example, the computing system may identify that one of the metrics in the collection of metrics 144, 146 relates to a time-domain HRV metric for a first REM sleep stage occurring during a sleep session. The computing system may access the sleep stage indicators 122 data to identify time stamps that identify the beginning and end of the first REM period of sleep. Using the time stamps, the computing system may select a portion of the metrics generated at box 420 that are specific to the first REM period. For example, FIG. 4 illustrates the REM period as being between minutes 184 and 232, for a total length of 48 minutes. The computing system may identify nine HRV metrics that were determined by the operations at box 420 and that relate to the first REM period of sleep.

In some examples, the identification of the relevant sleep stage and selection of data for the relevant sleep stage 430 is performed before one or more of the operations of boxes 410 and 420. For example, the system may only perform the operations of boxes 410 and 420 on portion of the heart waveform 104 that corresponds to the first REM sleep stage.

Although this description uses the first REM sleep stage as an example, the relevant sleep stage can be any single sleep stage during a sleep session (e.g., a particular N1 period, a particular N2 period, a particular N3 period, or a particular REM period), or a combination of multiple instances of a given type of sleep stage during a sleep session (e.g., all N1 periods, all N2 periods, all N3 periods, all REM periods).

At box 440, the computing system determines a time-domain HRV metric for a particular sleep stage by combining multiple time-domain HRV metrics for portions of the particular sleep stage. For example, the computing system may combine seven SDNN values computed for the seven respective 5-minute portions of time during the 48 minute REM time stage, for example, by averaging the seven SDNN values to generate a single averaged SDNN value. The single averaged SDNN represents the time-domain HRV metric for the first REM sleep stage in this example. The computing system may not select any sleep stage that does not last at least a certain number of epochs (e.g., excluding any sleep stage that does not last for at least ten five-minute epochs, for this time-domain HRV metric and for other metrics).

Time-domain HRV metrics may be based on, alternatively to or in addition to an SDNN value): (1) NN50 (e.g., number of adjacent NN intervals that differ by more than 50 ms), (2) pNN50 (e.g., percentage of adjacent NN intervals that differ from each other), (3) RMSSD (e.g., determined by taking the differences between normal heartbeats, squaring the differences, averaging the result, and take the square root of the averaged result), (4) HRMax−HRMin (e.g., average difference between the highest and lowest heart rates during a respiratory cycle), or (5) HRV triangular index (e.g., determined by taking an integral of the density of RR interval histogram, divided by height).

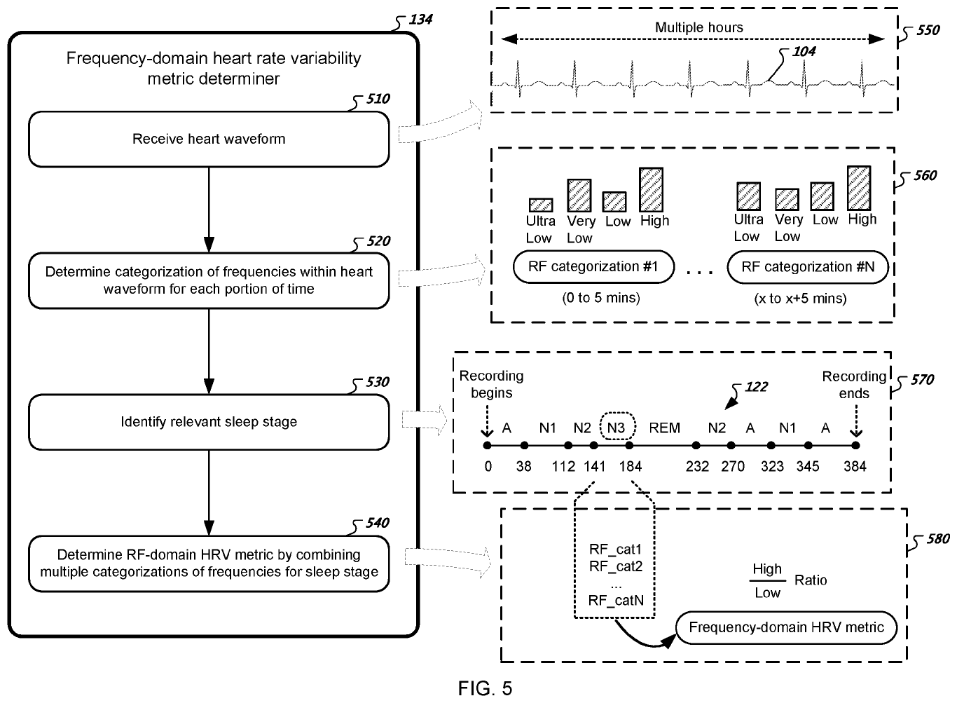

FIG. 5 shows the frequency-domain HRV metric determiner 134 of FIG. 1, with additional detail and illustration. The frequency-domain HRV metric determiner 134 can perform the operations of boxes 510-540 shown in FIG. 5 to generate a frequency-domain HRV metric that may be, or be used in the formation of, a metric included in the collection of metrics 144, 146.

At box 510, the computing system on which the frequency-domain HRV metric determiner 134 is operating can receive heart waveform data 104. The heart waveform 104 may represent multiple hours of heart operation of a patient, including a sleep session, as illustrated by item 550.

At box 520, the computing system determines a categorization of frequencies within the heart waveform for each portion of time. For example, the computing system may perform a Fast Fourier Transform on each five-minute portion of the heart waveform 104, to produce a categorization of frequencies specific to each respective five-minute portion, as illustrated by item 560. The categorization of frequencies may, for each of multiple frequency bands of the heart waveform, identify a level of energy within the respective frequency band over the respective period of time.

As an illustration, “RF categorization #1” (shorthand for “frequency categorization #1”) in item 560 FIG. 5 shows that the heart waveform for a first five-minute segment includes a relatively small component of Ultra Low frequencies (e.g., 0.003 Hz and below), a somewhat greater component of Low frequencies (e.g., 0.04-0.15 Hz), an even greater component of Very Low frequencies (e.g., 0.003-0.04 Hz), and a greatest component of High frequencies (e.g., 0.15-0.4 Hz). The processing of the RF categorizations can occur in real time during a sleep session as the heart waveform data 104 is sensed and processed, or after the sleep session during a post processing of sleep data. In some examples, intensities of Ultra Low frequency and Very Low frequency bands may be calculated over a longer time interval (e.g., multiple epochs, an entire sleep stage, or an entire sleep session).

At box 530, the computing system identifies the relevant sleep stage for which a metric is to be generated and selects data for the relevant sleep stage. For example, the computing system may identify that one of the metrics in the collection of metrics 144, 146 relates to a frequency-domain HRV metric for a first N3 sleep stage occurring during a sleep session. The computing system may access the sleep stage indicators 122 data to identify time stamps that identify the beginning and end of the first N3 period of sleep. Using the time stamps, the computing system may select a portion of the metrics generated at box 520 that are specific to the first N3 period. For example, FIG. 5 item 570 illustrates the first N3 period as being a 43 minute time period between 141 minutes and 184 minutes.

In some examples, the identification of the relevant sleep stage and selection of data for the relevant sleep stage 530 is performed before one or more of the operations of boxes 510 and 520. For example, the system may only perform the operations of boxes 510 and 520 on portion of the heart waveform 104 that corresponds to the first N3 sleep stage.

Although this description uses the first N3 sleep stage as an example, the relevant sleep stage can be any single sleep stage during a sleep session (e.g., a particular N1 period, a particular N2 period, a particular N3 period, or a particular REM period), or a combination of multiple instances of a given type of sleep stage during a sleep session (e.g., all N1 periods, all N2 periods, all N3 periods, all REM periods). To create a metric that indicates a ratio of High frequencies to Low frequencies during the first N3 sleep period, the computing system may select the first N3 sleep period as the relevant sleep stage.

At box 540, the computing system determines a frequency-domain HRV metric by combining multiple RF categorizations for portions of the particular sleep stage. For example, the computing system may combine eight RF categorizations computed for the eight five-minute segments of the 43 minute time period between 141 minutes and 184 minutes, by averaging the eight RF categorizations to generate a single averaged RF categorization, as illustrated by item 580.

The computing system may not consider sleep stages that do not last at least a certain number of epochs. Each component of an RF categorization may be an absolute number (e.g., a number for “Ultra Low” frequencies along a range from 0 to 1) or a relative number (e.g., a number for “Ultra Low” frequencies that represents a proportion of “Ultra Low” frequency components among all frequency components in a portion of the heart waveform 104).

In some examples, the computing system uses a value indicating an intensity of energy in a single frequency band to generate a metric (e.g., an intensity of “Low” frequencies during a particular sleep stage or other sleep period, without the value indicating intensities of other frequency bands).

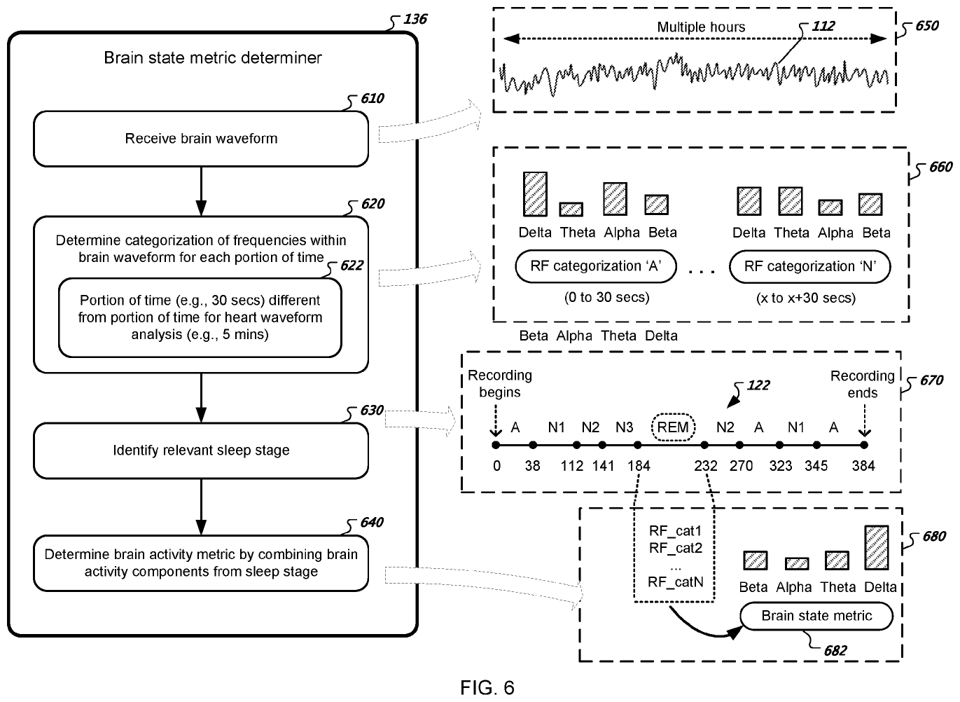

FIG. 6 shows the brain state metric determiner 136 of FIG. 1, with additional detail and illustration. The brain state metric determiner 136 can perform the operations of boxes 610-640 shown in FIG. 6 to generate a brain state metric that may be, or be used in the formation of, a metric included in the collection of metrics 144, 146.

At box 610, the computing system on which the brain state metric determiner 136 is operating can receive brain waveform data 102. The brain waveform data 102 may encode a waveform that represents multiple hours of brain operation of a patient, including during a sleep session, as illustrated by item 650.

At box 620, the computing system determines a categorization of frequencies within the brain waveform for each portion of time. For example, the computing system may perform a Fast Fourier Transform on each thirty-second portion of the brain waveform data 102, to produce a categorization of frequencies specific to each respective five-minute portion, as illustrated by item 660. The categorization of frequencies may, for each of multiple frequency bands of the brain waveform, identify a level of energy within the respective frequency band over the respective period of time.

As an illustration, “RF categorization A” in item 660 of FIG. 6 shows that the brain waveform for a first portion of time includes a relatively small component of Theta band frequencies (e.g., 4-8 Hz), a somewhat greater component of Beta band frequencies (e.g., 13-30 Hz), an even greater component of Alpha band frequencies (e.g., 8-12 Hz), and a greatest component of Delta band frequencies (e.g., less than 4 Hz). The processing of the RF categorizations can occur in real time during a sleep session as the brain waveform is sensed and processed, or after the sleep session during a post processing of sleep data.

At box 622, the portion of time (e.g., each 30 second epoch) for which each categorization of the brain waveform 102 is performed during creation of a brain state metric is different from the portion of time (e.g., each 5 minute epoch) for which each categorization of the heart waveform 104 is performed during creation of a heart-variability metric.

At box 630, the computing system identifies the relevant sleep stage which a metric is to be generated and selects data for the relevant sleep stage. For example, the computing system may identify that one of the metrics in the collection of metrics 144, 146 relates to a brain state metric for a first REM stage occurring during a sleep session. The computing system may access the sleep stage indicators 122 data to identify time stamps that identify the beginning and end of the first REM period of sleep. Using the time stamps, the computing system may select a portion of the metrics generated at box 620 that are specific to the first REM period. For example, FIG. 6 item 670 illustrates the first REM period as being a 48 minute time period between 184 minutes and 232 minutes.

In some examples, the identification of the relevant sleep stage and selection of data for the relevant sleep stage 630 is performed before one or more of the operations of boxes 610 and 620. For example, the system may only perform the operations of boxes 610 and 620 on portion of the brain waveform 102 that corresponds to the first REM sleep stage.

Although this description uses the first REM sleep stage as an example, the relevant sleep stage can be any single sleep stage during a sleep session (e.g., a particular N1 period, a particular N2 period, a particular N3 period, or a particular REM period), or a combination of multiple instances of a given type of sleep stage during a sleep session (e.g., all N1 periods, all N2 periods, all N3 periods, all REM periods).

At box 640, the computing system determines a brain state HRV metric 682 by combining multiple RF categorizations for portions of the particular sleep stage. For example, the computing system may combine ninety-six RF categorizations computed for the ninety-six 30-second intervals during the 48 minute time period between 184 minutes and 232 minutes to form the brain state metric 682, as illustrated by item 680. The combining may include averaging the ninety-six RF categorizations to generate a single averaged RF categorization.

The brain state metric 682 can be a portion of data selected from the averaged RF categorization or can be derived from such portion of data. For example, the brain state metric 682 can be or include the averaged RF categorization in its entirety, an amount of a certain band of waveforms from the averaged RF categorization, or a proportion of an amount a certain band of waveforms to an amount of another band of waveforms.

The computing system may not consider sleep stages that do not last at least a certain number of epochs. Further, each component of an RF categorization may be an absolute number (e.g., a number for “Beta” frequencies along a range from 0 to 1) or a relative number (e.g., a number for “Beta” frequencies that represents a proportion of “Beta” frequency components among all frequency components in a portion of the brain waveform 102).

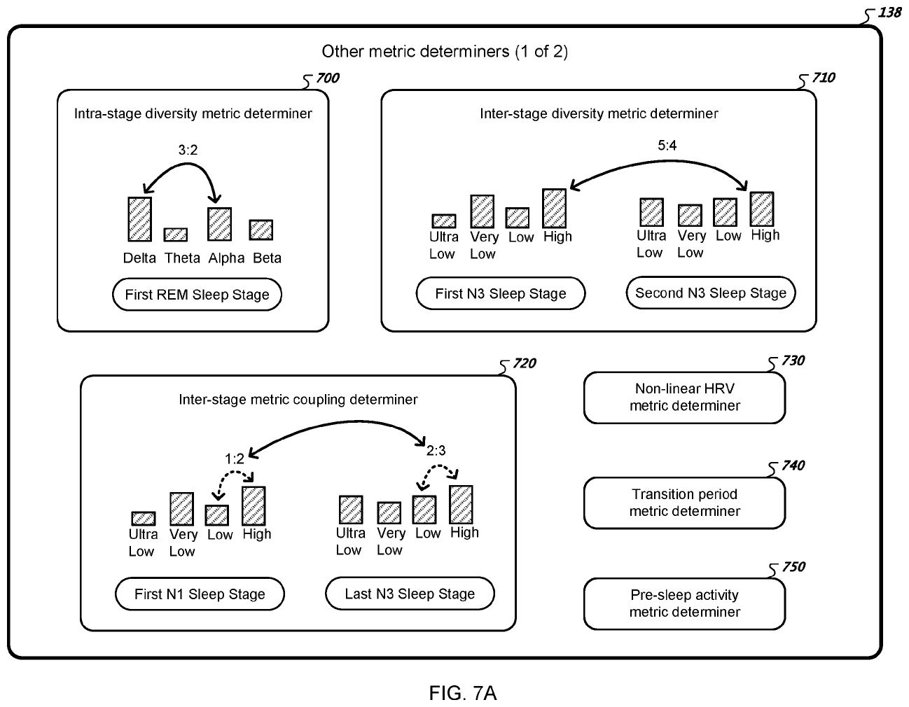

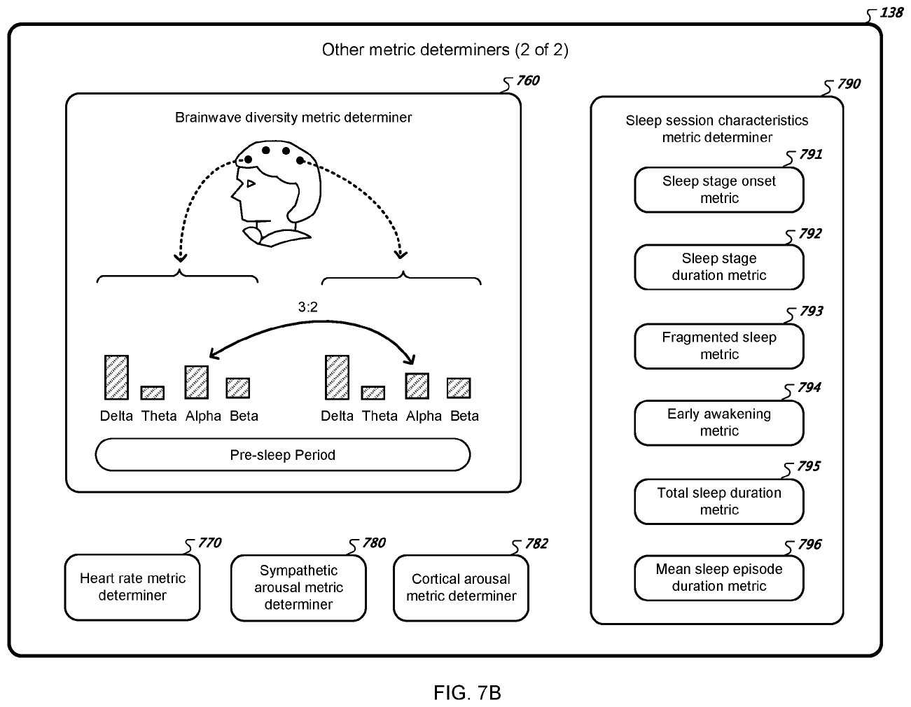

FIGS. 7A-B show the other metric determiners 138 of FIG. 1. Each of the other metric determiners 138 can represent algorithms executable by one or more devices to generate a metric that may be, or be used in the formation of, a metric included in the collection of metrics 144, 146.

An intra-stage diversity metric determiner 700 can generate a metric that indicates a relationship between values for two different metrics during the same sleep stage. For example, the determined metric may indicate a ratio between an intensity of delta brainwaves and alpha brain waves during a first REM sleep period of a sleep session (e.g., a ratio of 3:2, as illustrated by item 700 in FIG. 7A). The metrics may be based on any two metrics discussed in this disclosure, and the metric may be for any given sleep stage or combination of sleep stages. Another example metric indicates a ratio between low and high heart frequencies over a combination of all N3 and REM sleep stages in a sleep session.

An inter-stage diversity metric determiner 710 can generate a metric that indicates a relationship between values for one or more metrics over multiple different sleep stages. For example, the determined metric may indicate a ratio between an intensity of high frequency-domain heart frequencies during a first N3 sleep stage and a second N3 sleep stage (e.g., a ratio of 5:4, as illustrated by item 710 in FIG. 7A). The metric may be based on any two metrics discussed in this disclosure, and the metric may be for any given sleep stage or combination of sleep stages. In some examples, the metric for each sleep stage is different. For example, the determined metric may indicate a ratio between an intensity of delta brain waves during a first N3 sleep session and an intensity of saw tooth brain waves during a first REM sleep session. Another example metric is a difference between heart rate during a first REM sleep session and a last REM sleep session.

A metric coupling determiner 720 can generate a metric that indicates a relationship between a coupling of two metrics over multiple different sleep stages. For example, the determined metric may indicate how correlated the low and high frequency-domain heart frequencies are between the first N1 sleep stage and the last N3 sleep stage (e.g., does the ratio between the different frequency bands remain constant or differ from one sleep stage to a next), as illustrated by item 720 in FIG. 7A. The metric may not indicate an intensity of either metric, but rather whether the metrics change in similar proportions (e.g., both increase in intensity by 30% from one sleep stage to another).

A non-linear heart rate variability metric determiner 730 can perform operations to generate a non-linear HRV metric for each portion of heart waveform data 104 (e.g., each five-minute epoch), as described above with respect to the time-domain HRV metric determiner 132 and the frequency-domain HRV metric determiner 134. The non-linear HRV metric may represent an amount of non-deterministic (e.g., chaotic) heart operation within each given period of time. For example, the computing system may perform a detrended fluctuation analysis or a Poincare plot on each five-minute epoch of the heart waveform 104, and use the analysis/plot as the non-linear HRV statistic or derive a non-linear HRV statistic from each analysis/plot. The computing system may then combine (e.g., average) all non-linear HRV statistics from a relevant sleep stage, to generate a non-linear HRV metric specific to the relevant sleep stage.

A transition period metric determiner 740 can generate a metric that is based on physiological measurements recorded during a transition period between sleep stages. The metric can be (or be based on) any of the other metrics generated with physiological data from a transition period. For example, the metric may be (or be based on) a heart rate, an intensity of delta brainwaves, or a proportion of high-to-low heart frequencies for a combination of all N3-to-REM transitions. In some examples, the metric is (or is based) on a length of the metric (e.g., a length of the first REM-to-N3 transition, in number of 30-second epochs). The determination of the position and length of transition periods is discussed in additional detail with respect to FIGS. 2A-B.

A pre-sleep activity metric determiner 750 can generate a metric that is based on physiological measurements recorded before a patient falls asleep, with four example such pre-sleep periods illustrated in FIG. 3. The metric can be or be based on any of the other metrics, for a pre-sleep period. For example, the metric may indicate an overall intensity of all brainwave frequencies during the five-minute period before the patient first fell asleep. As another example, the metric may indicate a ratio of intensities of alpha brainwaves and beta brainwaves during a combination of all pre-sleep periods. As another example, the metric may indicate a frequency-domain heart rate during the first pre-sleep period.

A brainwave diversity metric determiner 760 (FIG. 7B) can generate a metric that is based on a relationship between brainwaves recorded by different electrodes. For example, the metric may indicate an amount of difference between a first EEG montage located on a first portion of the patient's head and a second EEG montage located on a second (different) portion of the patient's head. Individuals with certain mental disabilities may exhibit lateralization in brain activity that exceeds a threshold. For example, the metric may indicate an amount of difference between an intensity of the delta frequency band recorded by one or more electrodes connected to a left side of the patient's head and an intensity of the same delta frequency band recorded by one or more different electrodes connected to a right side of the patient's head. In another example, the metric may indicate a diversity between front and rear electrodes, as illustrated by item 760 in FIG. 7B.

A heart rate metric determiner 770 can generate a metric that is (or is based on) one or more of a mean, a median, an average, a variance, a skew, a kurtosis, a percentile, 5th and 95th percentiles, and a cumulative distribution function of a patient's heart rate, over an entire sleep session or a particular sleep stage. For example, the metric may be (or be based on) an average heart rate during REM sleep (e.g., the first REM session, or an average of all REM sleep stages, in a sleep session). In another example, the metric may be (or be based on) a difference in heart rate among sleep stages (e.g., an absolute or proportional difference between: (1) a mean heart rate during all REM sleep stages, and (2) a mean heart rate during all N3 sleep stages).

A sympathetic arousal metric determiner 780 can generate a metric that is (or is based on) a number of sympathetic arousals during a certain amount of time. For example, the metric may indicate a number of increases in heart rate or heart rate variability, measured as an increase of a threshold amount (e.g., percentage increase or threshold satisfied) within a certain amount of time (e.g., a certain number of heart beats, a number of minutes, a particular sleep stage, or an entire sleep session). The metric relates to an amount of times that brain activity prompts heart activity to increase during sleep.

A cortical arousal metric determiner 782 can generate a metric that is (or is based) on a number of cortical arousals during a certain amount of time. For example, the metric may indicate a number of increases in brainwave activity of any particular frequency band of brain activity, measured as increase of threshold amount (e.g., a percentage increase or threshold satisfied) within a certain amount of time (e.g., a certain number of heart beats, a number of minutes, a sleep stage, an entire sleep session).

A sleep session characteristics metric determiner 790 can generate any combination of multiple metrics that are based on characteristics of a sleep session. These metrics are described in additional detail below.

A sleep stage onset metric 791 can indicate an amount of time until a first occurrence of any one of N2 sleep, N3 sleep, and REM sleep. The amount of time may be calculated from measurements beginning, lights being turned off, or sleep onset (e.g., a beginning of N1 sleep).

A sleep stage duration metric 792 can indicate an amount of time in a sleep stage, as an absolute amount or as a proportion of a sleep stage with respect to another sleep stage or combination of sleep stages. For example, the metric can be based on an absolute amount of time in N1 sleep (e.g., 36 minutes over the entire sleep session). As another example, the metric can indicate a proportion of sleep that is REM sleep, calculated from time patient fell asleep until time patient last woke up (e.g., 9 percent).

A fragmented sleep metric 794 can indicate a quantity of times that a patient woke and fell back to sleep during a sleep session. For example, the metric can indicate that a patient woke up eleven times during a night.

An early awakening metric 795 can indicate whether a patient woke early and/or how early the patient woke. For example, the metric can indicate whether the patient woke for a final time before six hours of sleep had occurred (e.g., the patient only slept 5 hours and 23 minutes).

A total sleep duration metric 796 can indicate how much a patient slept during a sleep session. For example, the metric may be based on a value calculated as a sum of sleep stage lengths normalized by a length of the sleep session, with time awake not being a sleep stage for purposes of this calculation.

A sleep episode duration metric 797 can indicate a length of sleep stages. For example, the metric can indicate an average length of all REM sleep stages. As another example, the metric can indicate an average length of all sleep stages (e.g., N1, N2, N3, and REM).

Other metrics not illustrated in FIGS. 7A-B may include those that are (or are based on) any one of the following: (1) an amount of physical activity (e.g., how active a person is a week, or during a sleep session, as measured by a wearable device), (2) a number of times that sleep stage retrograded during a sleep session (e.g., a number of times that a sleep stage transitioned backwards to a lower sleep stage before REM sleep had been achieved for that sleep cycle), (3) non-physiological data 116, (4) a perspiration sensor (e.g., measured by a skin conductance sensor worn by a patient during a sleep session), (5) blood pressure data (e.g., measured by a blood pressure cuff worn by a patient during a sleep session), (6) blood sugar measured during sleep session, before the sleep session and/or after the sleep session, (7) a level of eye movement (e.g., as monitored by an image sensor contained in goggles of a headset worn during a sleep session), and (8) data from a computerized tomography scan and/or a magnetic resonance imaging scan.

The metric determiner 130 assembles multiple metrics for a patient, into a collection of metrics 144, 146, and can do this for each of many patients. A collection of metrics 144, 146 can include “N” different metrics in an ordered sequence. The collection of metrics 144, 146 for each respective patient may be the same, and may represent a vector of values (e.g., an array of eight values, each representing a single metric). Each metric in the collection 144 may have a same range (e.g., all represented by a number between 0 and 1), or at least some metrics may have different ranges (e.g., with a first metric represented by a value between 0 and 1, and a second metric represented by a value between 1 and 5).

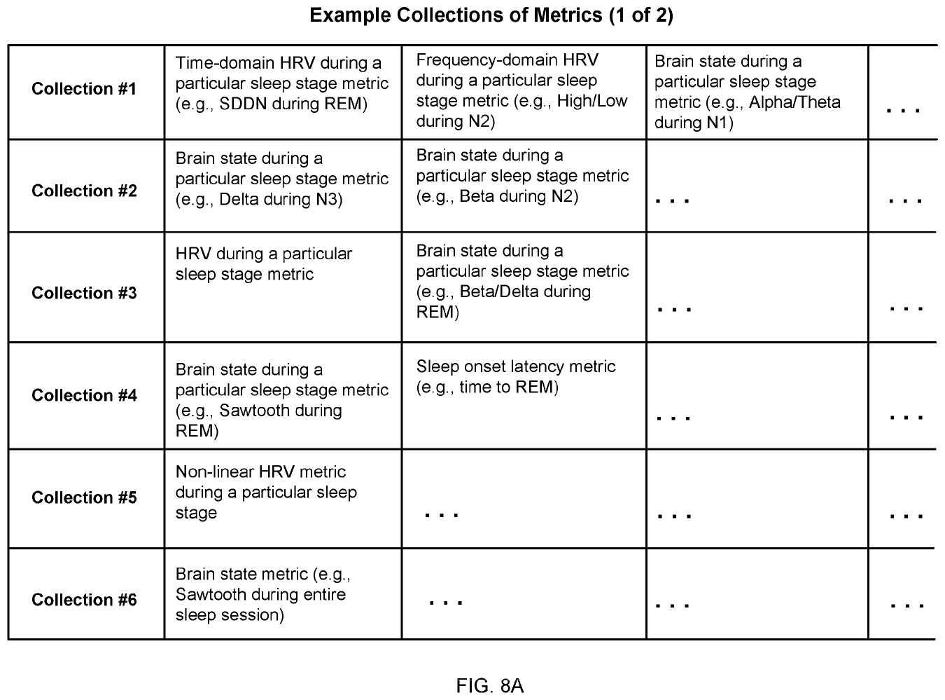

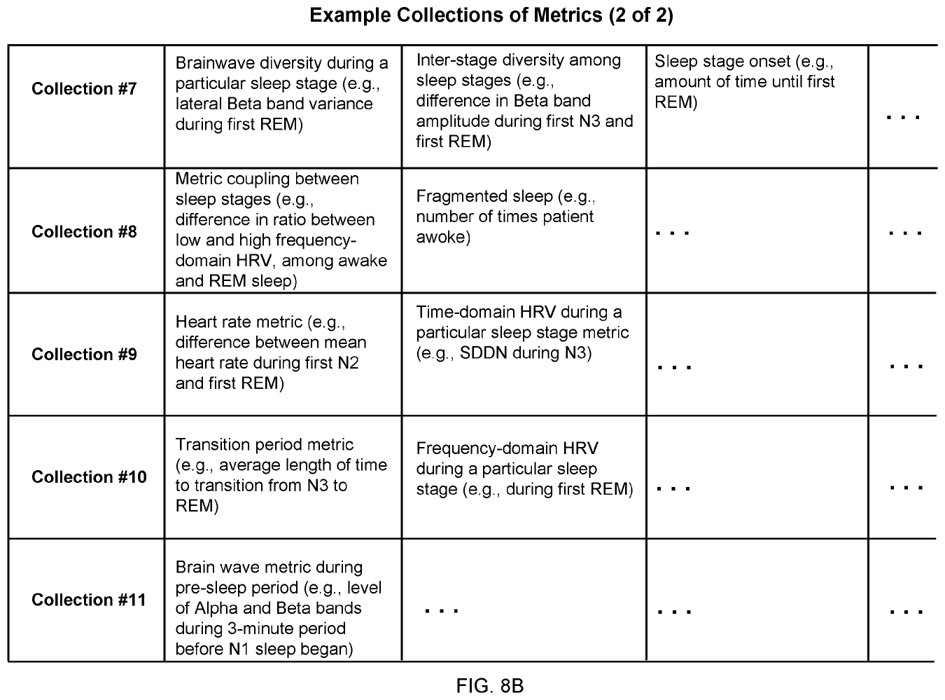

FIGS. 8A-B show multiple example collections of metrics. While any of the metrics described in this disclosure may be combined in any order to form a collection of metrics 144, 146, FIGS. 8A-B show eleven such example collections of metrics.

Collection #1 includes three or more metrics, including at least: (1) a time-domain heart-rate variability during a particular sleep stage metric (e.g., standard deviation of RR intervals during an REM sleep stage metric); (2) a frequency-domain heart-rate variability during a particular sleep stage metric (e.g., a ratio of high frequencies to low frequencies during an N2 sleep stage metric); and (3) a brain state during a particular sleep stage metric (e.g., a ratio of Alpha band frequencies to Theta band frequencies during an N1 sleep stage metric).

Collection #2 includes two or more metrics, including at least: (1) a brain state during a particular sleep stage metric (e.g., amount of Delta band frequencies during an N3 sleep stage metric); and (2) a brain state during a particular sleep stage metric (e.g., amount of delta band frequencies during an N3 sleep stage metric). As illustrated by Collection #2, a collection of metrics 144 may include multiple variations of a same type of metrics, such as an amount of the same brain state during different sleep stages, different brain states during the same sleep stage, and/or different brain states during different sleep stages. Different variations of each type of HRV metric may also be combined in a single collection of metrics 144.

Collection #3 includes two or more metrics, including at least: (1) a heart rate variability during a particular sleep stage metric (e.g., any of time-domain, frequency-domain, and non-linear HRV metrics during any particular sleep stage); and (2) a brain state during a particular sleep stage metric (e.g., a ratio of Beta band frequencies to Delta band frequencies during an REM sleep stage).

Collection #4 includes two or more metrics, including at least (1) a brain state during a particular sleep stage metric (e.g., an amount of Sawtooth band frequencies during an REM sleep stage metric); and (2) a sleep onset latency metric (e.g., a time from lights out until an REM sleep stage).

Collection #5 includes at least one metric, including at least (1) a non-linear heart-rate variability metric during a particular sleep stage (e.g., a result based on a detrended fluctuation analysis during a sleep stage that represents a combination of N3 and REM individual sleep stages).

Collection #6 includes at least one metric, including at least (1) a brain state metric (e.g., an amount of Sawtooth band frequencies during an entire sleep session, from falling asleep until awakening a final time).

Collection #7 (FIG. 8B) includes at least three metrics, including at least (1) a brainwave diversity during a particular sleep stage metric (e.g., a variation in beta frequency-band brainwave intensity between electrodes positioned at left and right sides of the patient's head, during a first REM sleep stage); (2) inter-stage diversity among sleep stages metric (e.g., a difference in beta frequency-band intensity during a first N3 sleep stage and a first REM sleep stage); and (3) sleep stage onset metric (e.g., an amount of time until first REM).

Collection #8 includes two or more metrics, including at least (1) a metric coupling between sleep stages metric (e.g., a difference in a ratio between low and high frequency-domain HRV, among an initial awake period and a first REM sleep stage); and (2) a fragmented sleep metric (e.g., an indication of a number of times that a patient woke up during a sleep session).

Collection #9 includes two or more metrics, including at least (1) a heart rate metric (e.g., a difference between mean heart rate during a first N2 sleep stage and a first REM sleep stage); and (2) a time-domain HRV during a particular sleep stage metric (e.g., SDDN during a last N3 sleep stage).

Collection #10 includes two or more metrics, including at least (1) a transition period metric (e.g., an average length of time to transition from N3 to REM sleep, based on all such instances occurring during a sleep session); and (2) an intensity of bands of a frequency-domain HRV during a particular sleep stage metric (e.g., intensity values for delta, theta, alpha, and beta brainwave bands during a first REM sleep stage of a sleep session).

Collection #11 includes one or more metrics, including at least (1) a brain wave during a pre-sleep period metric (e.g., intensities of alpha and beta brainwave bands during a 3-minute period immediately before a first N1 sleep stage began).

The collection of metrics 144 and 146 generated by the metric determiner (and on which the model trainer 150 and the mental state classifier 160 operate) may include one or more of the brain waveform 102, the heart waveform 104, the other patient data 106, and the non-physiological patient data 116. As such, the model trainer 150 and the mental state classifier 160 may operate on a collection of metrics that includes a brain waveform 102 without the heart waveform 104, the heart waveform 104 without the brain waveform 102, the other patient data 106 without the brain waveform 102 and without the heart waveform 104, or the non-physiological patient data 116 without any of the brain waveform 102, the heart waveform 106, and the other patient data 106.

The model trainer 150 (see FIG. 1B) receives multiple sets of: (1) a mental state classification 142 for a given patient, and (2) a collection of metrics 144 generated from analysis of physiological characteristics for the given patient, and uses this data to generate one or more trained computational models 152 (e.g., with each such trained model being trained with a different collection of metrics 144). Stated another way, the model trainer 150 receives, for each respective patient of multiple patients, a mental state classification 142 and a collection of metrics 144.

The mental state classification 142 for each patient can indicate whether the patient has been diagnosed as exhibiting a particular behavioral health state, such as whether the patient has been designated by a clinician as being depressed or exhibiting another behavioral health state (e.g., a mental disorder) for which the system has been trained. As such, the mental state classification 142 for each patient may be a binary value, with “1” indicating that the patient has been designated as exhibiting a particular mental state and “0” indicating that the patient has been designated as not exhibiting the particular mental state. In some examples, the mental state classification 142 may include multiple values representing a designation for the patient for each of multiple behavioral health states (e.g., depression, anxiety, and schizophrenia). In some examples, the mental state classification 142 may be a quantitative score that indicates a severity of a mental state (e.g., a severity of a patient's anxiety).

The nature of the computational model and the training performed can be of any appropriate form, and can include one or more of: decision tree learning, random forest, logistic regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, or the like.

Each trained model 152 generated by the model trainer 150 may be tested to assess the discriminatory performance of the trained model 152. Such testing may be performed using a subset of patient data (e.g., only the mental state classification 142 and collection of metrics 144 for some of the patients), and in particular, different patient data than that used to train the model, to avoid model bias. Discriminatory performance may be based on an accuracy, sensitivity, specificity, and/or AUROC (area under receiver characteristic operating curve), for example, with a discriminatory performance of at least 70% required in order for the model to be selected for use. As such, the above-described processes can be performed iteratively utilizing different collections of metrics and/or different computational models until a required degree of discriminatory power is obtained. Preliminary results based on cross-validation indicated a sensitivity of 71.7%, a specificity of 71.4%, a Positive Predictive Value of 35.4%, and a Negative Predictive Value of 92.1% when tested within a development sample.

After one or more trained models have been selected (e.g., by a human analyst or computer based on the one or more trained models exhibiting satisfactory discriminatory performance), a mental state classifier 160 may use the selected models-illustrated in FIG. 1 as a first trained model 162 and a second trained model 164. The dashed arrow from the trained computational models 152 to the mental state classifier 160 indicates that only one or more of the trained models may be selected for runtime mental state classification. The selected model(s) 152 may be used during to generate a mental state classification 170 for a patient for which a mental state is unknown, based upon a collection of metrics 146 generated from physiological and/or non-physiological patient data 116 of the patient.

For example, a patient for whom mental state is not known may participate in a sleep study, during which various biometrics are recorded, such as brain waveform data 102 and heart waveform data 104. The data processor 110, the sleep stage determiner 120, and the metric determiner 130 may operate upon such data in a same or similar manner as discussed above for the training process, in order to generate a collection of metrics 146. The collection of metrics 146 (generated for runtime classification) may represent a same ordered set of metrics as the collection of metrics 144 (generated for training) that were used to form the one or more selected computational models 152a-b. Still, the collection of metrics 146 (generated for runtime classification) may not be accompanied by a mental state classification 142, as with the collection of metrics 144 (generated for training).

The mental state classifier 160 may receive the collection of metrics 146 and input the collection of metrics 146 into a first trained model 162. In those implementations in which the mental state classifier 160 is configured to store and operate on more than one trained model (e.g., with FIG. 1 illustrating such a scenario, by depicting a second trained model 164), the mental state classifier 160 may receive more than one collection of metrics for inputting a collection of metrics into each respective trained model 152. In such implementations, each trained model may generate a classification and the classification combiner 166 may consider multiple such classifications to generate an overall mental state classification 170. The classification combiner 166, when implemented, may take a majority weight of results from trained models. More complex ensembling strategies may be used.

In examples in which the mental state classifier 160 uses a single trained computational model 152, there may be no need for the classification combiner 166, and the mental state classification may be an output of a single trained computational model 152.

The mental state classification 170 can represent a likelihood of a patient having a mental state and/or a severity of such a mental state, depending upon implementation of the system 100 and nature of the mathematical model(s) used (e.g., whether more than trained model is used, and a type of the trained model). The mental state classification 170 can indicate a likelihood or severity of any of a variety of mental states, such as normal and abnormal mental states, as well as specific conditions such as depression, anxiety, panic disorder, obsessive compulsive disorder, and schizophrenia.

The output of each trained model and/or the output of the mental state classifier 160 may be, for example: (1) a range of classifications (e.g., a number between 0.00 and 1.00); or (2) a binary output based on a range of classifications (e.g., 0 or 1, based on rounding values in a range between 1.0 and 5.0 up or down). For example, the mental state classification 170 can include a numerical value within a range, for example indicating that the user has a 95% likelihood of suffering from depression.

The computing system 100 can provide an indication of the mental state classification 170 for presentation to a clinician or the patient on an appropriate display device (e.g., a desktop computer at which the clinician receives results, or a smartphone of the patient in an email or an application program that provided the physiological data to a cloud server system for analysis).

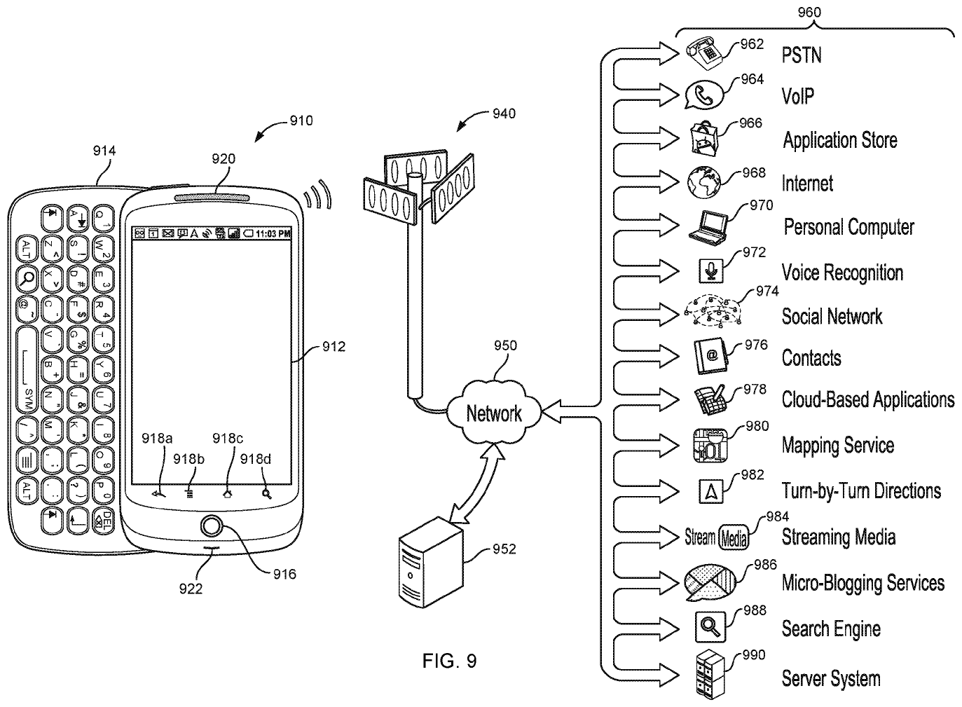

Referring now to FIG. 9, a conceptual diagram of a system that may be used to implement the systems and methods described in this document is illustrated. In the system, mobile computing device 910 can wirelessly communicate with base station 940, which can provide the mobile computing device wireless access to numerous hosted services 960 through a network 950.

In this illustration, the mobile computing device 910 is depicted as a handheld mobile telephone (e.g., a smartphone, or an application telephone) that includes a touchscreen display device 912 for presenting content to a user of the mobile computing device 910 and receiving touch-based user inputs and/or presence-sensitive user input (e.g., as detected over a surface of the computing device using radar detectors mounted in the mobile computing device 510). Other visual, tactile, and auditory output components may also be provided (e.g., LED lights, a vibrating mechanism for tactile output, or a speaker for providing tonal, voice-generated, or recorded output), as may various different input components (e.g., keyboard 914, physical buttons, trackballs, accelerometers, gyroscopes, and magnetometers).

Example visual output mechanism in the form of display device 912 may take the form of a display with resistive or capacitive touch capabilities. The display device may be for displaying video, graphics, images, and text, and for coordinating user touch input locations with the location of displayed information so that the device 910 can associate user contact at a location of a displayed item with the item. The mobile computing device 910 may also take alternative forms, including as a laptop computer, a tablet or slate computer, a personal digital assistant, an embedded system (e.g., a car navigation system), a desktop personal computer, or a computerized workstation.

An example mechanism for receiving user-input includes keyboard 914, which may be a full qwerty keyboard or a traditional keypad that includes keys for the digits ‘0-9’, ‘*’, and ‘#.’ The keyboard 914 receives input when a user physically contacts or depresses a keyboard key. User manipulation of a trackball 916 or interaction with a track pad enables the user to supply directional and rate of movement information to the mobile computing device 910 (e.g., to manipulate a position of a cursor on the display device 912).

The mobile computing device 910 may be able to determine a position of physical contact with the touchscreen display device 912 (e.g., a position of contact by a finger or a stylus). Using the touchscreen 912, various “virtual” input mechanisms may be produced, where a user interacts with a graphical user interface element depicted on the touchscreen 912 by contacting the graphical user interface element. An example of a “virtual” input mechanism is a “software keyboard,” where a keyboard is displayed on the touchscreen and a user selects keys by pressing a region of the touchscreen 912 that corresponds to each key.

The mobile computing device 910 may include mechanical or touch sensitive buttons 918a-d. Additionally, the mobile computing device may include buttons for adjusting volume output by the one or more speakers 920, and a button for turning the mobile computing device on or off. A microphone 922 allows the mobile computing device 910 to convert audible sounds into an electrical signal that may be digitally encoded and stored in computer-readable memory, or transmitted to another computing device. The mobile computing device 910 may also include a digital compass, an accelerometer, proximity sensors, and ambient light sensors.

An operating system may provide an interface between the mobile computing device's hardware (e.g., the input/output mechanisms and a processor executing instructions retrieved from computer-readable medium) and software. Example operating systems include ANDROID, CHROME, IOS, MAC OS X, WINDOWS 7, WINDOWS PHONE 7, SYMBIAN, BLACKBERRY, WEBOS, a variety of UNIX operating systems; or a proprietary operating system for computerized devices. The operating system may provide a platform for the execution of application programs that facilitate interaction between the computing device and a user.

The mobile computing device 910 may present a graphical user interface with the touchscreen 912. A graphical user interface is a collection of one or more graphical interface elements and may be static (e.g., the display appears to remain the same over a period of time), or may be dynamic (e.g., the graphical user interface includes graphical interface elements that animate without user input).

A graphical interface element may be text, lines, shapes, images, or combinations thereof. For example, a graphical interface element may be an icon that is displayed on the desktop and the icon's associated text. In some examples, a graphical interface element is selectable with user-input. For example, a user may select a graphical interface element by pressing a region of the touchscreen that corresponds to a display of the graphical interface element. In some examples, the user may manipulate a trackball to highlight a single graphical interface element as having focus. User-selection of a graphical interface element may invoke a pre-defined action by the mobile computing device. In some examples, selectable graphical interface elements further or alternatively correspond to a button on the keyboard 914. User-selection of the button may invoke the pre-defined action.

In some examples, the operating system provides a “desktop” graphical user interface that is displayed after turning on the mobile computing device 910, after activating the mobile computing device 910 from a sleep state, after “unlocking” the mobile computing device 910, or after receiving user-selection of the “home” button 918c. The desktop graphical user interface may display several graphical interface elements that, when selected, invoke corresponding application programs. An invoked application program may present a graphical interface that replaces the desktop graphical user interface until the application program terminates or is hidden from view.

User-input may influence an executing sequence of mobile computing device 910 operations. For example, a single-action user input (e.g., a single tap of the touchscreen, swipe across the touchscreen, contact with a button, or combination of these occurring at a same time) may invoke an operation that changes a display of the user interface. Without the user-input, the user interface may not have changed at a particular time. For example, a multi-touch user input with the touchscreen 912 may invoke a mapping application to “zoom-in” on a location, even though the mapping application may have by default zoomed-in after several seconds.

The desktop graphical interface can also display “widgets.” A widget is one or more graphical interface elements that are associated with an application program that is executing, and that display on the desktop content controlled by the executing application program. A widget's application program may launch as the mobile device turns on. Further, a widget may not take focus of the full display. Instead, a widget may only “own” a small portion of the desktop, displaying content and receiving touchscreen user-input within the portion of the desktop.

The mobile computing device 910 may include one or more location-identification mechanisms. A location-identification mechanism may include a collection of hardware and software that provides the operating system and application programs an estimate of the mobile device's geographical position. A location-identification mechanism may employ satellite-based positioning techniques, base station transmitting antenna identification, multiple base station triangulation, internet access point IP location determinations, inferential identification of a user's position based on search engine queries, and user-supplied identification of location (e.g., by receiving user a “check in” to a location).

The mobile computing device 910 may include other applications, computing sub-systems, and hardware. A call handling unit may receive an indication of an incoming telephone call and provide a user the capability to answer the incoming telephone call. A media player may allow a user to listen to music or play movies that are stored in local memory of the mobile computing device 910. The mobile computing device 910 may include a digital camera sensor, and corresponding image and video capture and editing software. An internet browser may enable the user to view content from a web page by typing in an addresses corresponding to the web page or selecting a link to the web page.

The mobile computing device 910 may include an antenna to wirelessly communicate information with the base station 940. The base station 940 may be one of many base stations in a collection of base stations (e.g., a mobile telephone cellular network) that enables the mobile computing device 910 to maintain communication with a network 950 as the mobile computing device is geographically moved. The computing device 910 may alternatively or additionally communicate with the network 950 through a Wi-Fi router or a wired connection (e.g., ETHERNET, USB, or FIREWIRE). The computing device 910 may also wirelessly communicate with other computing devices using BLUETOOTH protocols, or may employ an ad-hoc wireless network.

A service provider that operates the network of base stations may connect the mobile computing device 910 to the network 950 to enable communication between the mobile computing device 910 and other computing systems that provide services 960. Although the services 960 may be provided over different networks (e.g., the service provider's internal network, the Public Switched Telephone Network, and the Internet), network 950 is illustrated as a single network. The service provider may operate a server system 952 that routes information packets and voice data between the mobile computing device 910 and computing systems associated with the services 960.

The network 950 may connect the mobile computing device 910 to the Public Switched Telephone Network (PSTN) 962 in order to establish voice or fax communication between the mobile computing device 910 and another computing device. For example, the service provider server system 952 may receive an indication from the PSTN 962 of an incoming call for the mobile computing device 910. Conversely, the mobile computing device 910 may send a communication to the service provider server system 952 initiating a telephone call using a telephone number that is associated with a device accessible through the PSTN 962.

The network 950 may connect the mobile computing device 910 with a Voice over Internet Protocol (VOIP) service 964 that routes voice communications over an IP network, as opposed to the PSTN. For example, a user of the mobile computing device 910 may invoke a VoIP application and initiate a call using the program. The service provider server system 952 may forward voice data from the call to a VoIP service, which may route the call over the internet to a corresponding computing device, potentially using the PSTN for a final leg of the connection.

An application store 966 may provide a user of the mobile computing device 910 the ability to browse a list of remotely stored application programs that the user may download over the network 950 and install on the mobile computing device 910. The application store 966 may serve as a repository of applications developed by third-party application developers. An application program that is installed on the mobile computing device 910 may be able to communicate over the network 950 with server systems that are designated for the application program. For example, a VoIP application program may be downloaded from the Application Store 966, enabling the user to communicate with the VoIP service 964.

The mobile computing device 910 may access content on the internet 968 through network 950. For example, a user of the mobile computing device 910 may invoke a web browser application that requests data from remote computing devices that are accessible at designated universal resource locations. In various examples, some of the services 960 are accessible over the internet.

The mobile computing device may communicate with a personal computer 970. For example, the personal computer 970 may be the home computer for a user of the mobile computing device 910. Thus, the user may be able to stream media from his personal computer 970. The user may also view the file structure of his personal computer 970, and transmit selected documents between the computerized devices.

A voice recognition service 972 may receive voice communication data recorded with the mobile computing device's microphone 922, and translate the voice communication into corresponding textual data. In some examples, the translated text is provided to a search engine as a web query, and responsive search engine search results are transmitted to the mobile computing device 910.

The mobile computing device 910 may communicate with a social network 974. The social network may include numerous members, some of which have agreed to be related as acquaintances. Application programs on the mobile computing device 910 may access the social network 974 to retrieve information based on the acquaintances of the user of the mobile computing device. For example, an “address book” application program may retrieve telephone numbers for the user's acquaintances. In various examples, content may be delivered to the mobile computing device 910 based on social network distances from the user to other members in a social network graph of members and connecting relationships. For example, advertisement and news article content may be selected for the user based on a level of interaction with such content by members that are “close” to the user (e.g., members that are “friends” or “friends of friends”).

The mobile computing device 910 may access a personal set of contacts 976 through network 950. Each contact may identify an individual and include information about that individual (e.g., a phone number, an email address, and a birthday). Because the set of contacts is hosted remotely to the mobile computing device 910, the user may access and maintain the contacts 976 across several devices as a common set of contacts.

The mobile computing device 910 may access cloud-based application programs 978. Cloud-computing provides application programs (e.g., a word processor or an email program) that are hosted remotely from the mobile computing device 910, and may be accessed by the device 910 using a web browser or a dedicated program. Example cloud-based application programs include GOOGLE DOCS word processor and spreadsheet service, GOOGLE GMAIL webmail service, and PICASA picture manager.

Mapping service 980 can provide the mobile computing device 910 with street maps, route planning information, and satellite images. An example mapping service is GOOGLE MAPS. The mapping service 980 may also receive queries and return location-specific results. For example, the mobile computing device 910 may send an estimated location of the mobile computing device and a user-entered query for “pizza places” to the mapping service 980. The mapping service 980 may return a street map with “markers” superimposed on the map that identify geographical locations of nearby “pizza places.”

Turn-by-turn service 982 may provide the mobile computing device 910 with turn-by-turn directions to a user-supplied destination. For example, the turn-by-turn service 982 may stream to device 910 a street-level view of an estimated location of the device, along with data for providing audio commands and superimposing arrows that direct a user of the device 910 to the destination.

Various forms of streaming media 984 may be requested by the mobile computing device 910. For example, computing device 910 may request a stream for a pre-recorded video file, a live television program, or a live radio program. Example services that provide streaming media include YOUTUBE and PANDORA.

A micro-blogging service 986 may receive from the mobile computing device 910 a user-input post that does not identify recipients of the post. The micro-blogging service 986 may disseminate the post to other members of the micro-blogging service 986 that agreed to subscribe to the user.

A search engine 988 may receive user-entered textual or verbal queries from the mobile computing device 910, determine a set of internet-accessible documents that are responsive to the query, and provide to the device 910 information to display a list of search results for the responsive documents. In examples where a verbal query is received, the voice recognition service 972 may translate the received audio into a textual query that is sent to the search engine.

These and other services may be implemented in a server system 990. A server system may be a combination of hardware and software that provides a service or a set of services. For example, a set of physically separate and networked computerized devices may operate together as a logical server system unit to handle the operations necessary to offer a service to hundreds of computing devices. A server system is also referred to herein as a computing system.

In various implementations, operations that are performed “in response to” or “as a consequence of” another operation (e.g., a determination or an identification) are not performed if the prior operation is unsuccessful (e.g., if the determination was not performed). Operations that are performed “automatically” are operations that are performed without user intervention (e.g., intervening user input). Features in this document that are described with conditional language may describe implementations that are optional. In some examples, “transmitting” from a first device to a second device includes the first device placing data into a network for receipt by the second device, but may not include the second device receiving the data. Conversely, “receiving” from a first device may include receiving the data from a network, but may not include the first device transmitting the data.

“Determining” by a computing system can include the computing system requesting that another device perform the determination and supply the results to the computing system. Moreover, “displaying” or “presenting” by a computing system can include the computing system sending data for causing another device to display or present the referenced information.

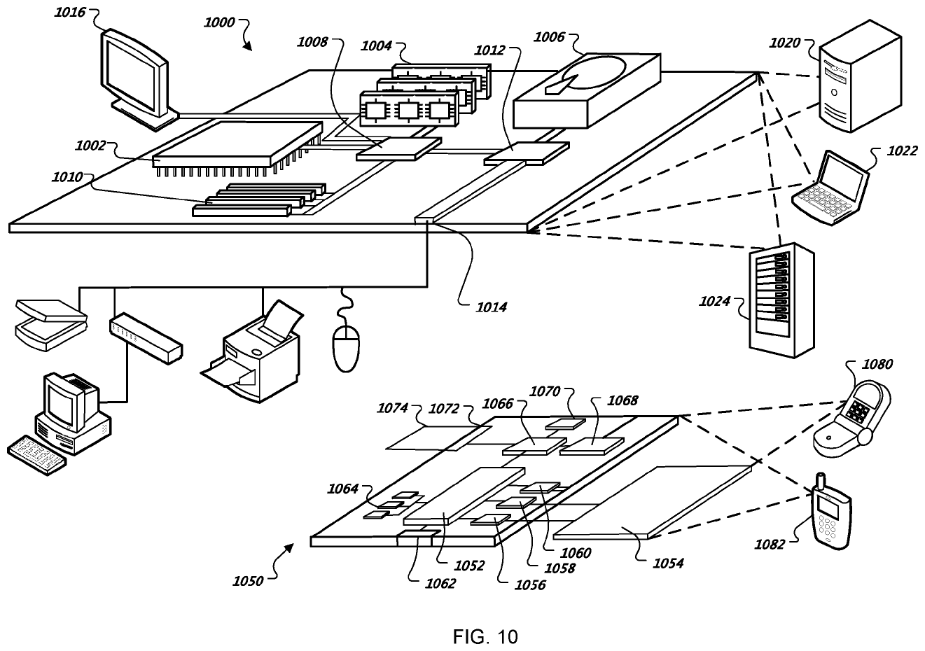

FIG. 10 is a block diagram of computing devices 1000, 1050 that may be used to implement the systems and methods described in this document, as either a client or as a server or plurality of servers. Computing device 1000 is intended to represent various forms of digital computers, such as laptops, desktops, workstations, personal digital assistants, servers, blade servers, mainframes, and other appropriate computers. Computing device 1050 is intended to represent various forms of mobile devices, such as personal digital assistants, cellular telephones, smartphones, and other similar computing devices. The components shown here, their connections and relationships, and their functions, are meant to be examples only, and are not meant to limit implementations described and/or claimed in this document.

Computing device 1000 includes a processor 1002, memory 1004, a storage device 1006, a high-speed controller 1008 connecting to memory 1004 and high-speed expansion ports 1010, and a low speed controller 1012 connecting to low speed expansion port 1014 and storage device 1006. Each of the components 1002, 1004, 1006, 1008, 1010, and 1012, are interconnected using various busses, and may be mounted on a common motherboard or in other manners as appropriate. The processor 1002 can process instructions for execution within the computing device 1000, including instructions stored in the memory 1004 or on the storage device 1006 to display graphical information for a GUI on an external input/output device, such as display 1016 coupled to high-speed controller 1008. In other implementations, multiple processors and/or multiple buses may be used, as appropriate, along with multiple memories and types of memory. Also, multiple computing devices 1000 may be connected, with each device providing portions of the necessary operations (e.g., as a server bank, a group of blade servers, or a multi-processor system).

The memory 1004 stores information within the computing device 1000. In one implementation, the memory 1004 is a volatile memory unit or units. In another implementation, the memory 1004 is a non-volatile memory unit or units. The memory 1004 may also be another form of computer-readable medium, such as a magnetic or optical disk.

The storage device 1006 is capable of providing mass storage for the computing device 1000. In one implementation, the storage device 1006 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer-or machine-readable medium, such as the memory 1004, the storage device 1006, or memory on processor 1002.

The high-speed controller 1008 manages bandwidth-intensive operations for the computing device 1000, while the low speed controller 1012 manages lower bandwidth-intensive operations. Such allocation of functions is an example only. In one implementation, the high-speed controller 1008 is coupled to memory 1004, display 1016 (e.g., through a graphics processor or accelerator), and to high-speed expansion ports 1010, which may accept various expansion cards (not shown). In the implementation, low-speed controller 1012 is coupled to storage device 1006 and low-speed expansion port 1014. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 1000 may be implemented in a number of different forms, as shown in the figure. For example, it may be implemented as a standard server 1020, or multiple times in a group of such servers. It may also be implemented as part of a rack server system 1024. In addition, it may be implemented in a personal computer such as a laptop computer 1022. Alternatively, components from computing device 1000 may be combined with other components in a mobile device (not shown), such as device 1050. Each of such devices may contain one or more of computing device 1000, 1050, and an entire system may be made up of multiple computing devices 1000, 1050 communicating with each other.

Computing device 1050 includes a processor 1052, memory 1064, an input/output device such as a display 1054, a communication interface 1066, and a transceiver 1068, among other components. The device 1050 may also be provided with a storage device, such as a microdrive or other device, to provide additional storage. Each of the components 1050, 1052, 1064, 1054, 1066, and 1068, are interconnected using various buses, and several of the components may be mounted on a common motherboard or in other manners as appropriate.

The processor 1052 can execute instructions within the computing device 1050, including instructions stored in the memory 1064. The processor may be implemented as a chipset of chips that include separate and multiple analog and digital processors. Additionally, the processor may be implemented using any of a number of architectures. For example, the processor may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 1050, such as control of user interfaces, applications run by device 1050, and wireless communication by device 1050.

Processor 1052 may communicate with a user through control interface 1058 and display interface 1056 coupled to a display 1054. The display 1054 may be, for example, a TFT (Thin-Film-Transistor Liquid Crystal Display) display or an OLED (Organic Light Emitting Diode) display, or other appropriate display technology. The display interface 1056 may comprise appropriate circuitry for driving the display 1054 to present graphical and other information to a user. The control interface 1058 may receive commands from a user and convert them for submission to the processor 1052. In addition, an external interface 1062 may be provide in communication with processor 1052, so as to enable near area communication of device 1050 with other devices. External interface 1062 may provided, for example, for wired communication in some implementations, or for wireless communication in other implementations, and multiple interfaces may also be used.

The memory 1064 stores information within the computing device 1050. The memory 1064 can be implemented as one or more of a computer-readable medium or media, a volatile memory unit or units, or a non-volatile memory unit or units. Expansion memory 1074 may also be provided and connected to device 1050 through expansion interface 1072, which may include, for example, a SIMM (Single In Line Memory Module) card interface. Such expansion memory 1074 may provide extra storage space for device 1050, or may also store applications or other information for device 1050. Specifically, expansion memory 1074 may include instructions to carry out or supplement the processes described above, and may include secure information also. Thus, for example, expansion memory 1074 may be provide as a security module for device 1050, and may be programmed with instructions that permit secure use of device 1050. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer-or machine-readable medium, such as the memory 1064, expansion memory 1074, or memory on processor 1052 that may be received, for example, over transceiver 1068 or external interface 1062.

Device 1050 may communicate wirelessly through communication interface 1066, which may include digital signal processing circuitry where necessary. Communication interface 1066 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1068. In addition, short-range communication may occur, such as using a Bluetooth, WiFi, or other such transceiver (not shown). In addition, GPS (Global Positioning System) receiver module 1070 may provide additional navigation-and location-related wireless data to device 1050, which may be used as appropriate by applications running on device 1050.

Device 1050 may also communicate audibly using audio codec 1060, which may receive spoken information from a user and convert it to usable digital information. Audio codec 1060 may likewise generate audible sound for a user, such as through a speaker, e.g., in a handset of device 1050. Such sound may include sound from voice telephone calls, may include recorded sound (e.g., voice messages, music files, etc.) and may also include sound generated by applications operating on device 1050.

The computing device 1050 may be implemented in a number of different forms, as shown in the figure. For example, it may be implemented as a cellular telephone 1080. It may also be implemented as part of a smartphone 1082, personal digital assistant, or other similar mobile device.

Additionally computing device 1000 or 1050 can include Universal Serial Bus (USB) flash drives. The USB flash drives may store operating systems and other applications. The USB flash drives can include input/output components, such as a wireless transmitter or USB connector that may be inserted into a USB port of another computing device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor. and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

As additional description to the embodiments described above, the present disclosure describes the following embodiments.

Embodiment 1 is a computer-implemented method, comprising: identifying, by a computing system, a value of a heart rate variability metric that indicates a variation in a heart waveform of a patient; identifying, by the computing system, a value of a brain activity metric that indicates a type of electrical activity represented by a brain waveform of the patient; providing, by the computing system, values for a collection of metrics to a computational model, the values for the collection of metrics including the value for the heart rate variability metric and the value for the brain activity metric; and receiving, by the computing system from the computational model as a result of having provided the values for the collection of metrics to the computational model, an indication of mental state of the patient.

Embodiment 2 is the computer-implemented method of embodiment 1, wherein the computational model comprises a machine learning model that has been trained.

Embodiment 3 is the computer-implemented method of embodiment 2, comprising: training, by the computing system, the machine learning model by providing training data that includes, for each respective patient of multiple patients: (i) a respective value of the heart rate variability metric for the respective patient; (ii) a respective value of the brain activity metric for the respective patient; and (iii) a respective indication of mental state for the respective patient.

Embodiment 4 is the computer-implemented method of any one of embodiments 1-3, wherein: the heart waveform comprises a waveform from an electrocardiogram of the patient; and the brain waveform comprises a waveform from an electroencephalogram of the patient.

Embodiment 5 is the computer-implemented method of any one of embodiments 1-4, comprising: determining, by the computing system, a value for a time-domain heart rate variability metric that indicates variance among lengths of heart beat intervals over a period of time in the heart waveform of the patient, wherein the value for the heart rate variability metric comprises the value for the time-domain heart rate variability metric.

Embodiment 6 is the computer-implemented method of embodiment 5,wherein the period of time is a combination of all instances of REM sleep stage during a sleep session.

Embodiment 7 is the computer-implemented method of any one of embodiments 1-6, comprising: determining, by the computing system, a value for a frequency-domain heart rate variability metric that indicates a categorization of frequencies within the heart waveform of the patient over a period of time into a collection of different heart beat frequency ranges, wherein the value for the heart rate variability metric comprises the value for the frequency-domain heart rate variability metric.

Embodiment 8 is the computer-implemented method of embodiment 7, wherein the period of time is a particular sleep stage of the patient, such that the frequency-domain heart rate variability metric does not indicate categorization of frequencies within a sleep stage other than the particular sleep stage.

Embodiment 9 is the computer-implemented method of embodiment 8, wherein the particular sleep stage is a first N3 sleep stage or a first REM sleep stage of a sleep session.

Embodiment 10 is the computer-implemented method of any one of embodiments 8-9, wherein: the frequency-domain heart rate variability metric is determined by combining: (i) a first categorization of frequencies within the heart waveform of the patient over a first portion of the period of time, with a second categorization of frequencies within the heart waveform of the patient over a second portion of the period of time; and the first portion of the period of time and the second portion of the period of time are a same length of time.

Embodiment 11 is the computer-implemented method of any one of embodiments 8-10, wherein the categorization of frequencies within the heart waveform of the patient over the period of time into the collection of different heart beat frequency ranges indicates intensities for each frequency range within the collection of different heart beat frequency ranges.

Embodiment 12 is the computer-implemented method of any one of embodiments 1-11, comprising: determining, by the computing system, a value for a non-linear heart rate variability metric that indicates an amount of non-linear variability over a period of time in the heart waveform of the patient, wherein the value for the heart rate variability metric comprises the value for the non-linear heart rate variability metric.

Embodiment 13 is the computer-implemented method of any one of embodiments 1-12, comprising: determining, by the computing system, a value for a brain state metric that indicates an amount of the electrical activity represented by the brain waveform of the patient that falls into a particular frequency band from among a collection of multiple different frequency bands, wherein the value for the brain activity metric comprises the value for the brain state metric.

Embodiment 14 is the computer-implemented method of embodiment 13, wherein the brain state metric indicates the amount of electrical activity that falls into the particular frequency band within a particular sleep stage of the patient, such that the brain state metric does not indicate electrical activity that falls within a sleep stage other than the particular sleep stage.

Embodiment 15 is the computer-implemented method of embodiment 14, wherein the particular sleep stage is a first N3 sleep stage or a first REM sleep stage of a sleep session.

Embodiment 16 is the computer-implemented method of any one of embodiments 14-15, determining, by the computing system, a value for a frequency-domain heart rate variability metric that indicates a categorization of frequencies within the heart waveform of the patient over a second sleep stage that is different from the particular sleep stage, wherein the value for the heart rate variability metric comprises the value for the frequency-domain heart rate variability metric.

Embodiment 17 is the computer-implemented method of embodiment 16, wherein the frequency-domain heart rate variability metric does not indicate categorization of frequencies within a sleep stage other than the second sleep stage.

Embodiment 18 is the computer-implemented method of any one of embodiments 1-17, wherein: the values for the collection of metrics provided to the computational model and on which the indication of the metal state of the patient is based includes a value for a sleep onset metric that indicates an amount of time before the patient experienced a particular sleep stage.

Embodiment 19 is the computer-implemented method of embodiment 18, comprising: determining, by the computing system based on analysis of the brain waveform of the patient: (i) a starting time within the brain waveform at which the patient began experiencing the particular sleep stage; and (ii) an ending time within the brain waveform at which the patient stopped experiencing the particular sleep stage.

Embodiment 20 is the computer-implemented method of embodiment 19, wherein the particular sleep stage is a first N3 sleep stage or a first REM sleep stage of a sleep session.

Embodiment 21 is the computer-implemented method of any one of embodiments 19-20, wherein the amount of time before the patient experienced the particular sleep stage represents an amount of time between the patient being determined to have fallen asleep and the patient beginning to experience the particular sleep stage.

Embodiment 22 is the computer-implemented method of any one of embodiments 1-21, comprising: determining, by the computing system, a value for a heart rate frequency metric that indicates an intensity of a range of frequencies in the heart waveform of the patient over a period of time, wherein the values for the collection of metrics that are provided to the computational model and on which the indication of the metal state of the patient is based includes the value for the heart rate frequency metric.

Embodiment 23 is the computer-implemented method of any one of embodiments 1-22, comprising: determining, by the computing system, a value for an intra-stage diversity metric that indicates a ratio between: (i) an intensity of brain activity or heart activity within a first frequency band during an instance of a particular sleep stage from the brain waveform or the heart waveform of the patient; and (ii) an intensity of brain activity or heart activity within a second frequency band during the instance of the particular sleep stage from the brain waveform or the heart waveform of the patient, wherein the value for the brain state metric or the heart rate variability metric comprises the intra-stage diversity metric.

Embodiment 24 is the computer-implemented method of any one of embodiments 1-23, comprising: determining, by the computing system, a value for an inter-stage diversity metric that indicates a ratio between: (i) an intensity of brain activity or heart activity within a particular frequency band during a first instance of a particular sleep stage from the brain waveform or the heart waveform of the patient; and (ii) an intensity of brain activity or heart activity within the particular frequency band during a second instance of the particular sleep stage from the brain waveform or the heart waveform of the patient, wherein the value for the brain state metric or the heart rate metric comprises the value for the inter-stage diversity metric.

Embodiment 25 is the computer-implemented method of any one of embodiments 1-24, comprising: determining, by the computing system, a value for a brainwave diversity metric that indicates a difference between: (i) an intensity of brain activity recorded by a first electrode on a first side of a head of the patient; and (ii) an intensity of brain activity recorded by a second electrode on a second side of the head of the patient opposite the first side of the head of the patient, wherein the value for the brain state metric comprises the value for the brainwave diversity metric.

Embodiment 26 is the computer-implemented method of any one of embodiments 1-25, comprising: determining, by the computing system, a value for an inter-stage coupling metric that indicates a coupling between: (i) a ratio between a first metric and a second metric during a first sleep stage; and (ii) a ratio between the first metric and the second metric during a second sleep stage, wherein the values for the collection of metrics that are provided to the computational model and on which the indication of the metal state of the patient is based include the value for the inter-stage metric coupling metric.

Embodiment 27 is the computer-implemented method of embodiment 26, wherein: the first sleep stage is a first instance of a particular sleep stage; and the second sleep stage is a second instance of the particular sleep stage.

Embodiment 28 is the computer-implemented method of any one of embodiments 1-27, comprising: identifying, by the computing system, physiological data recorded from the patient while the patient was in a transitional period between sleep stages; and determining, by the computing system, a value for a transition-specific metric that indicates a value for another type of metric during the transitional period between sleep stages, and wherein the values for the collection of metrics that are provided to the computational model and on which the indication of the metal state of the patient is based include the value for the transition-specific metric.

Embodiment 29 is the computer-implemented method of any one of embodiments 1-28, comprising: identifying, by the computing system, physiological data recorded from the patient during a period of time that preceded the patient falling asleep; and determining, by the computing system, a value for a pre-sleep activity metric that indicates a value for another type of metric during the period of time that preceded the patient falling asleep, and wherein the values for the collection of metrics that are provided to the computational model and on which the indication of the metal state of the patient is based include the value for the pre-sleep activity metric.

Embodiment 30 is a computing system, comprising: one or more processors; and one or more computer-readable devices including instructions that, when executed by the one or more processors, cause the computing system to perform the method of any one of embodiments 1-29.

Embodiment 31 is a computer-implemented method, comprising: identifying, by a computing system, a value of a heart rate variability metric that indicates a variation in a heart waveform of a patient; providing, by the computing system, values for a collection of metrics to a computational model, the values for the collection of metrics including the value for the heart rate variability metric; and receiving, by the computing system from the computational model as a result of having provided the values for the collection of metrics to the computational model, an indication of mental state of the patient.

Embodiment 32 is a computer-implemented method, comprising: identifying, by a computing system, a value of a brain activity metric that indicates a type of electrical activity represented by a brain waveform of the patient; providing, by the computing system, values for a collection of metrics to a computational model, the values for the collection of metrics including the value for the brain activity metric; and receiving, by the computing system from the computational model as a result of having provided the values for the collection of metrics to the computational model, an indication of mental state of the patient.

Embodiment 33 is a computer-implemented method, comprising: receiving, by a computing system, data that was generated from an analysis of one or more document that indicate characteristics of a patient during a sleep study, the data indicating a value for a particular metric; providing, by the computing system, values for a collection of metrics to a computational model, the values for the collection of metrics including the value for the particular metric generated from the analysis of one or more documents that indicate characteristics of the patient during the sleep study; and receiving, by the computing system from the computational model as a result of having provided the values for the collection of metrics to the computational model, an indication of mental state of the patient.

Embodiment 34 is a computing system, comprising: one or more processors; and one or more computer-readable devices including instructions that, when executed by the one or more processors, cause the computing system to perform the method of any one of embodiments 31-33.

Although a few implementations have been described in detail above, other modifications are possible. Moreover, other mechanisms for performing the systems and methods described in this document may be used. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following claims.

1. A computer-implemented method, comprising:
identifying, by a computing system, a value of a heart rate variability metric that indicates a variation in a heart waveform of a patient;
identifying, by the computing system, a value of a brain activity metric that indicates a type of electrical activity represented by a brain waveform of the patient;
providing, by the computing system, values for a collection of metrics to a computational model, the values for the collection of metrics including the value for the heart rate variability metric and the value for the brain activity metric; and
receiving, by the computing system from the computational model as a result of having provided the values for the collection of metrics to the computational model, an indication of mental state of the patient.

2. The computer-implemented method of claim 1, wherein the computational model comprises a machine learning model that has been trained.

3. The computer-implemented method of claim 2, comprising:
training, by the computing system, the machine learning model by providing training data that includes, for each respective patient of multiple patients:
(i) a respective value of the heart rate variability metric for the respective patient;
(ii) a respective value of the brain activity metric for the respective patient; and
(iii) a respective indication of mental state for the respective patient.

4. The computer-implemented method of claim 1, wherein:
the heart waveform comprises a waveform from an electrocardiogram of the patient; and
the brain waveform comprises a waveform from an electroencephalogram of the patient.

5. The computer-implemented method of claim 1, comprising:
determining, by the computing system, a value for a time-domain heart rate variability metric that indicates variance among lengths of heart beat intervals over a period of time in the heart waveform of the patient,
wherein the value for the heart rate variability metric comprises the value for the time-domain heart rate variability metric.

6. The computer-implemented method of claim 5, wherein the period of time is a combination of all instances of REM sleep stage during a sleep session.

7. The computer-implemented method of claim 1, comprising:
determining, by the computing system, a value for a frequency-domain heart rate variability metric that indicates a categorization of frequencies within the heart waveform of the patient over a period of time into a collection of different heart beat frequency ranges,
wherein the value for the heart rate variability metric comprises the value for the frequency-domain heart rate variability metric.

8. The computer-implemented method of claim 7, wherein the period of time is a particular sleep stage of the patient, such that the frequency-domain heart rate variability metric does not indicate categorization of frequencies within a sleep stage other than the particular sleep stage.

9. The computer-implemented method of claim 8, wherein the particular sleep stage is a first N3 sleep stage or a first REM sleep stage of a sleep session.

10. The computer-implemented method of claim 8, wherein:
the frequency-domain heart rate variability metric is determined by combining: (i) a first categorization of frequencies within the heart waveform of the patient over a first portion of the period of time, with a second categorization of frequencies within the heart waveform of the patient over a second portion of the period of time; and
the first portion of the period of time and the second portion of the period of time are a same length of time.

11. The computer-implemented method of claim 8, wherein the categorization of frequencies within the heart waveform of the patient over the period of time into the collection of different heart beat frequency ranges indicates intensities for each frequency range within the collection of different heart beat frequency ranges.

12. The computer-implemented method of claim 1, comprising:
determining, by the computing system, a value for a non-linear heart rate variability metric that indicates an amount of non-linear variability over a period of time in the heart waveform of the patient,
wherein the value for the heart rate variability metric comprises the value for the non-linear heart rate variability metric.

13. The computer-implemented method of claim 1, comprising:
determining, by the computing system, a value for a brain state metric that indicates an amount of the electrical activity represented by the brain waveform of the patient that falls into a particular frequency band from among a collection of multiple different frequency bands,
wherein the value for the brain activity metric comprises the value for the brain state metric.

14. The computer-implemented method of claim 13, wherein the brain state metric indicates the amount of electrical activity that falls into the particular frequency band within a particular sleep stage of the patient, such that the brain state metric does not indicate electrical activity that falls within a sleep stage other than the particular sleep stage.

15. The computer-implemented method of claim 14, wherein the particular sleep stage is a first N3 sleep stage or a first REM sleep stage of a sleep session.

16. The computer-implemented method of claim 14, determining, by the computing system, a value for a frequency-domain heart rate variability metric that indicates a categorization of frequencies within the heart waveform of the patient over a second sleep stage that is different from the particular sleep stage,
wherein the value for the heart rate variability metric comprises the value for the frequency-domain heart rate variability metric.

17. The computer-implemented method of claim 16, wherein the frequency-domain heart rate variability metric does not indicate categorization of frequencies within a sleep stage other than the second sleep stage.

18. The computer-implemented method of claim 1, wherein:
the values for the collection of metrics provided to the computational model and on which the indication of the metal state of the patient is based includes a value for a sleep onset metric that indicates an amount of time before the patient experienced a particular sleep stage.

19. The computer-implemented method of claim 18, comprising:
determining, by the computing system based on analysis of the brain waveform of the patient:
(i) a starting time within the brain waveform at which the patient began experiencing the particular sleep stage; and
(ii) an ending time within the brain waveform at which the patient stopped experiencing the particular sleep stage.

20. The computer-implemented method of claim 19, wherein the particular sleep stage is a first N3 sleep stage or a first REM sleep stage of a sleep session.

21. The computer-implemented method of claim 19, wherein the amount of time before the patient experienced the particular sleep stage represents an amount of time between the patient being determined to have fallen asleep and the patient beginning to experience the particular sleep stage.

22. The computer-implemented method of claim 1, comprising:
determining, by the computing system, a value for a heart rate frequency metric that indicates an intensity of a range of frequencies in the heart waveform of the patient over a period of time,
wherein the values for the collection of metrics that are provided to the computational model and on which the indication of the metal state of the patient is based includes the value for the heart rate frequency metric.

23. The computer-implemented method of claim 1, comprising:
determining, by the computing system, a value for an intra-stage diversity metric that indicates a ratio between:
(i) an intensity of brain activity or heart activity within a first frequency band during an instance of a particular sleep stage from the brain waveform or the heart waveform of the patient; and
(ii) an intensity of brain activity or heart activity within a second frequency band during the instance of the particular sleep stage from the brain waveform or the heart waveform of the patient,
wherein the value for the brain state metric or the heart rate variability metric comprises the intra-stage diversity metric.

24. The computer-implemented method of claim 1, comprising:
determining, by the computing system, a value for an inter-stage diversity metric that indicates a ratio between:
(i) an intensity of brain activity or heart activity within a particular frequency band during a first instance of a particular sleep stage from the brain waveform or the heart waveform of the patient; and
(ii) an intensity of brain activity or heart activity within the particular frequency band during a second instance of the particular sleep stage from the brain waveform or the heart waveform of the patient,
wherein the value for the brain state metric or the heart rate metric comprises the value for the inter-stage diversity metric.

25. The computer-implemented method of claim 1, comprising:
determining, by the computing system, a value for a brainwave diversity metric that indicates a difference between:
(i) an intensity of brain activity recorded by a first electrode on a first side of a head of the patient; and
(ii) an intensity of brain activity recorded by a second electrode on a second side of the head of the patient opposite the first side of the head of the patient,
wherein the value for the brain state metric comprises the value for the brainwave diversity metric.

26. The computer-implemented method of claim 1, comprising:
determining, by the computing system, a value for an inter-stage coupling metric that indicates a coupling between:
(i) a ratio between a first metric and a second metric during a first sleep stage; and
(ii) a ratio between the first metric and the second metric during a second sleep stage,
wherein the values for the collection of metrics that are provided to the computational model and on which the indication of the metal state of the patient is based include the value for the inter-stage metric coupling metric.

27. The computer-implemented method of claim 26, wherein:
the first sleep stage is a first instance of a particular sleep stage; and
the second sleep stage is a second instance of the particular sleep stage.

28. The computer-implemented method of claim 1, comprising:
identifying, by the computing system, physiological data recorded from the patient while the patient was in a transitional period between sleep stages; and
determining, by the computing system, a value for a transition-specific metric that indicates a value for another type of metric during the transitional period between sleep stages, and
wherein the values for the collection of metrics that are provided to the computational model and on which the indication of the metal state of the patient is based include the value for the transition-specific metric.

29. The computer-implemented method of claim 1, comprising:
identifying, by the computing system, physiological data recorded from the patient during a period of time that preceded the patient falling asleep; and
determining, by the computing system, a value for a pre-sleep activity metric that indicates a value for another type of metric during the period of time that preceded the patient falling asleep, and
wherein the values for the collection of metrics that are provided to the computational model and on which the indication of the metal state of the patient is based include the value for the pre-sleep activity metric.

30. A computing system, comprising:
one or more processors; and
one or more computer-readable devices including instructions that, when executed by the one or more processors, cause the computing system to perform operations that include:
identifying, by the computing system, a value of a heart rate variability metric that indicates a variation in a heart waveform of a patient;
identifying, by the computing system, a value of a brain activity metric that indicates a type of electrical activity represented by a brain waveform of the patient;
providing, by the computing system, values for a collection of metrics to a computational model, the values for the collection of metrics including the value for the heart rate variability metric and the value for the brain activity metric; and
receiving, by the computing system from the computational model as a result of having provided the values for the collection of metrics to the computational model, an indication of mental state of the patient.
